SEQUENCE LISTING (56 sequences)

SEQ ID NO: 1 - DNA, 20 bp, Artificial Sequence
  Synthesized
gcagaaggcctcagcaccta

SEQ ID NO: 2 - DNA, 19 bp, Artificial Sequence
  Synthesized
aggttcccagtcgggttca

SEQ ID NO: 3 - DNA, 20 bp, Artificial Sequence
  Synthesized
gactttaactggagcacaga

SEQ ID NO: 4 - DNA, 20 bp, Artificial Sequence
  Synthesized
agctttattagggatggcaa

SEQ ID NO: 5 - DNA, 26 bp, Artificial Sequence
  Synthesized
tgggagcggtgaagatggaagggcac

SEQ ID NO: 6 - DNA, 26 bp, Artificial Sequence
  Synthesized
tcatgccagcgcccacgtacgacgac

SEQ ID NO: 7 - DNA, 26 bp, Artificial Sequence
  Synthesized
cgctttcatggtgtgggctaaggacg

SEQ ID NO: 8 - DNA, 26 bp, Artificial Sequence
  Synthesized
tagttggggtggtcctgcatgtgctg

SEQ ID NO: 9 - DNA, 21 bp, Artificial Sequence
  Synthesized
gcccacacaccacttacttcg

SEQ ID NO: 10 - DNA, 21 bp, Artificial Sequence
  Synthesized
gtccgtcaggtaagcagggac

SEQ ID NO: 11 - DNA, 21 bp, Artificial Sequence
  Synthesized
ggccaagtacatcccagcttt

SEQ ID NO: 12 - DNA, 19 bp, Artificial Sequence
  Synthesized
cagcaccagctcgtcaagg

SEQ ID NO: 13 - DNA, 20 bp, Artificial Sequence
  Synthesized
ggaagtcggtgaagaacggg

SEQ ID NO: 14 - DNA, 20 bp, Artificial Sequence
  Synthesized
tgttggagatgacgtcgctg

SEQ ID NO: 15 - DNA, 20 bp, Artificial Sequence
  Synthesized
cgcggcgtatccgtgtcctc

SEQ ID NO: 16 - DNA, 24 bp, Artificial Sequence
  Synthesized
agcctgttccgtctcaaacttggt

SEQ ID NO: 17 - DNA, 21 bp, Artificial Sequence
  Synthesized
tttatgccccggaactccttt

SEQ ID NO: 18 - DNA, 21 bp, Artificial Sequence
  Synthesized
agtctctgtttggcagacgaa

SEQ ID NO: 19 - DNA, 19 bp, Artificial Sequence
  Synthesized
tgggagccatttgcctctg

SEQ ID NO: 20 - DNA, 21 bp, Artificial Sequence
  Synthesized
agccgtggtggaataggagta

SEQ ID NO: 21 - DNA, 20 bp, Artificial Sequence
  Synthesized
gagctgctccatctgtaggg

SEQ ID NO: 22 - DNA, 20 bp, Artificial Sequence
  Synthesized
cacagtctgctgaggttgga

SEQ ID NO: 23 - DNA, 20 bp, Artificial Sequence
  Synthesized
gagttctccgtggacgagac

SEQ ID NO: 24 - DNA, 23 bp, Artificial Sequence
  Synthesized
tccagtggtcatcatttcctttc

SEQ ID NO: 25 - DNA, 24 bp, Artificial Sequence
  Synthesized
cttcatccaatggactgcataaat

SEQ ID NO: 26 - DNA, 28 bp, Artificial Sequence
  Synthesized
tcccaagtataacactctacacagacaa

SEQ ID NO: 27 - DNA, 20 bp, Artificial Sequence
  Synthesized
aaacttggccgtggaaacct

SEQ ID NO: 28 - DNA, 21 bp, Artificial Sequence
  Synthesized
cagcataggctgttgacagtc

SEQ ID NO: 29 - DNA, 20 bp, Artificial Sequence
  Synthesized
cttcgctacctgcctaaccc

SEQ ID NO: 30 - DNA, 21 bp, Artificial Sequence
  Synthesized
gactgtgtcaaatcctgctcc

SEQ ID NO: 31 - DNA, 18 bp, Artificial Sequence
  Synthesized
cagcacttcctgaatgag

SEQ ID NO: 32 - DNA, 23 bp, Artificial Sequence
  Synthesized
aggtgactgggaggacttgaggc

SEQ ID NO: 33 - DNA, 20 bp, Artificial Sequence
  Synthesized
ttgaagcctctcgttgaccc

SEQ ID NO: 34 - DNA, 18 bp, Artificial Sequence
  Synthesized
cgtggtgggatacagcaa

SEQ ID NO: 35 - DNA, 23 bp, Artificial Sequence
  Synthesized
aatctgtacctgtcctgcgtgtt

SEQ ID NO: 36 - DNA, 24 bp, Artificial Sequence
  Synthesized
tgggtaatttttgggatctacact

SEQ ID NO: 37 - DNA, 21 bp, Artificial Sequence
  Synthesized
ggtacatcctcgacggcatct

SEQ ID NO: 38 - DNA, 21 bp, Artificial Sequence
  Synthesized
gtgcctctttgctgctttcac

SEQ ID NO: 39 - DNA, 20 bp, Artificial Sequence
  Synthesized
cttggcagccttcctgattt

SEQ ID NO: 40 - DNA, 23 bp, Artificial Sequence
  Synthesized
ttctttagcactccttggcaaaa

SEQ ID NO: 41 - DNA, 20 bp, Artificial Sequence
  Synthesized
gcctaacatgcttcgagatc

SEQ ID NO: 42 - DNA, 20 bp, Artificial Sequence
  Synthesized
tgatgtctgggtcttggttc

SEQ ID NO: 43 - DNA, 20 bp, Artificial Sequence
  Synthesized
ggagaagggtgaccgactca

SEQ ID NO: 44 - DNA, 17 bp, Artificial Sequence
  Synthesized
ctgcccagactcggcaa

SEQ ID NO: 45 - DNA, 19 bp, Artificial Sequence
  Synthesized
tctggaatgggaagacacc

SEQ ID NO: 46 - DNA, 19 bp, Artificial Sequence
  Synthesized
cacatctgcccagtagtgc

SEQ ID NO: 47 - DNA, 20 bp, Artificial Sequence
  Synthesized
agatgcagcctcatttccac

SEQ ID NO: 48 - DNA, 20 bp, Artificial Sequence
  Synthesized
gccttggatggaagaacaaa

SEQ ID NO: 49 - DNA, 23 bp, Artificial Sequence
  Synthesized
tcagcgaggcggctttggagcag

SEQ ID NO: 50 - DNA, 21 bp, Artificial Sequence
  Synthesized
catgtcttcgatgtcggtcag

SEQ ID NO: 51 - DNA, 20 bp, Artificial Sequence
  Synthesized
cctaccacgggtggatgttc

SEQ ID NO: 52 - DNA, 20 bp, Artificial Sequence
  Synthesized
caacatgggttttcggcctg

SEQ ID NO: 53 - DNA, 20 bp, Artificial Sequence
  Synthesized
agcttggtggtggatgaaac

SEQ ID NO: 54 - DNA, 20 bp, Artificial Sequence
  Synthesized
ccctcttcagcaaagcagac

SEQ ID NO: 55 - DNA, 21 bp, Artificial Sequence
  Synthesized
ggccaagtacatcccagcttt

SEQ ID NO: 56 - DNA, 19 bp, Artificial Sequence
  Synthesized
cagcaccagctcgtcaagg

(19) United States
(12) Patent Application Publication
(10) Pub. No.: US 2022/0308045 A1
(43) Pub. Date: Sep. 29, 2022

(54) EXPANDABLE LIVER ORGANOIDS, MEDIA COMPOSITION FOR DIFFERENTIATION THEREOF, AND METHOD FOR PRODUCING LIVER ORGANOIDS USING THE SAME

(71) Applicant: KOREA RESEARCH INSTITUTE OF BIOSCIENCE AND BIOTECHNOLOGY, Daejeon (KR)

(72) Inventors: Myung Jin SON, Daejeon (KR); Kyung-Sook CHUNG, Daejeon (KR); Seonju MUN, Daejeon (KR); Jae Sung RYU, Daejeon (KR); Cho-Rok JUNG, Daejeon (KR); Hyun-Soo CHO, Daejeon (KR); Dae Soo KIM, Daejeon (KR)

(21) Appl. No.: 17/686,669

(22) Filed: Mar. 4, 2022

Related U.S. Application Data
(63) Continuation-in-part of application No. PCT/KR2020/009035, filed on Jul. 9, 2020.
Application No. PCT/KR2020/009035 is a continuation-in-part of application No. PCT/KR2020/009033, filed on Jul. 9, 2020.

(30) Foreign Application Priority Data
Sep. 4, 2019 (KR) 10-2019-0109378

(51) Int. Cl. G01N 33/50; G01N 33/68; C12Q 1/6851; C12N 5/071

(52) U.S. Cl. CPC G01N 33/5082; G01N 33/6893; C12Q 1/6851; C12N 5/0671; C12N 2501/115; C12N 2500/25; C12N 2501/237; C12N 2506/45; C12N 2501/12; C12N 2501/11; C12Q 2600/158

(57) ABSTRACT

The present invention relates to expandable liver organoids, a medium composition for differentiation thereof, and a method for producing liver organoids using the same, and the liver organoids according to the present invention exhibit the characteristics of more mature hepatocytes than 2D differentiated hepatocytes, can be subcultured up to 90 times or more, and exhibit the expandability for maintaining the characteristics of mature hepatocytes even after multiple subcultures, and thus can be usefully utilized for predicting toxicity, regeneration, and inflammatory response, drug screening, and modeling of diseases such as hepatic steatosis.

### CROSS-REFERENCE TO RELATED APPLICATION

This application is a Continuation-In-Part (CIP) of International Application No. PCT/KR2020/009035, filed Jul. 9, 2020 and International Application No. PCT/KR2020/009033, filed Jul. 9, 2020, which are based on and claim priority to Korean Patent Application No. 10-2019-0109378 filed on Sep. 4, 2019. All the aforementioned applications are hereby incorporated by reference in their entireties.

### TECHNICAL FIELD

The present invention relates to expandable liver organoids, a medium composition for differentiation thereof, and a method for producing liver organoids using the same.

### BACKGROUND ART

Human cell-based and personalized in-vitro liver models are required for drug efficacy and toxicity tests in preclinical drug development. A liver is a representative organ having inherent regenerative potential in vivo, but there is a limitation in that primary human hepatocytes (PHHs), which are considered as the gold standard for evaluating liver metabolism, lose proliferation ability and organ functionality in vitro.

In order to overcome this limitation of PHHs, various approaches have been developed, including genetic modification, three-dimensional (3D) culturing combined with tissue engineering technology, and defined medium composition. However, the development of alternative and sustainable cell sources to reproduce inherent liver function remains a challenge.

Meanwhile, stem cells are a useful alternative source of hepatocytes, and the hepatocytes can be obtained in various ways from pluripotent stem cells (PSCs). Liver spheroids or organoids generated from PSCs have been attracting attention as stem cell-based in vitro 3D liver models, but it is difficult to maintain proliferation activity and functionality. Another alternative, tissue-derived liver organoids have limitations in terms of accessibility to human tissues and their narrow potential for differentiation.

Therefore, there is a need for the development of a method capable of generating expandable and more mature liver organoids derived from PSCs including human embryonic stem cells (hESCs) and induced pluripotent stem cells (iPSCs).

The present inventors have made intensive efforts to solve the above problems, and thus have confirmed that the liver organoids of the present invention exhibit a more mature phenotype compared with 2D differentiated hepatocytes, can be subcultured up to 90 times or more, and maintain the characteristics of the hepatocytes even after multiple subcultures, and completed the present invention. Therefore, the present invention provides human liver organoids suitable for predicting toxicity, regeneration, and inflammatory response, drug screening, and modeling of diseases such as hepatic steatosis.

### DISCLOSURE OF INVENTION

### Technical Problem

An object of the present invention is to provide a medium composition for differentiation of liver organoids that can be subcultured up to 90 times or more and maintain characteristics of mature hepatocytes even through multiple subcultures.

Another object of the present invention is to provide a method for producing the liver organoids using the medium composition.

Still another object of the present invention is to provide liver organoids produced by the method.

Yet still another object of the present invention is to provide liver organoids that express particular liver-specific genes, can be subcultured up to 90 times or more, and maintain the characteristics of mature hepatocytes even through multiple subcultures.

Another object of the present invention is to provide a method for screening hepatotoxic drugs using the liver organoids.

Still another object of the present invention is to provide a method for screening therapeutic agents for fatty liver.

### Solution to Problem

To solve the problems, the present invention provides a medium composition for differentiation of liver organoids, the medium composition including a basic fibroblast growth factor (bFGF), oncostatin M (OSM), and insulin-transferrin-selenium (ITS).

The present invention also provides a method for producing liver organoids, the method including culturing, in the medium composition, hepatic endoderm cells, or hepatocytes which are differentiated from stem cells.

The present invention also provides liver organoids produced by the method.

The present invention also provides liver organoids that express, as a liver-specific genetic marker, AMBP, APOA2, APOB, CYP8B1, F2, FGA, FGB, FGG, HABP2, ITIH2, PROC, SERPINA11, SERPINA4, SLC2A2, UGT2B15, and VTN.

The present invention also provides a method for screening hepatotoxic drugs, the method including contacting a test substance with the liver organoids, and measuring cell viability or an oxygen consumption rate (OCR) in the liver organoids.

The present invention also provides a method for screening therapeutic agents for fatty liver, the method including producing the liver organoids into fatty liver organoids, and treating the fatty liver organoids with candidate materials for therapeutic agents for fatty liver.

### Advantageous Effects of Invention

The liver organoids of the present invention exhibit the characteristics of more mature hepatocytes than 2D differentiated hepatocytes, are easily obtained as compared with tissue-derived liver organoids, can be subcultured up to 90 times or more, and exhibit the expandability for maintaining the characteristics of mature hepatocytes even after multiple subcultures, and thus can be usefully utilized for predicting toxicity, regeneration, and inflammatory response, drug screening, and modeling of diseases such as hepatic steatosis.

### BRIEF DESCRIPTION OF DRAWINGS

FIG. 1 is a schematic diagram illustrating a process of producing liver organoids from pluripotent stem cells.

FIG. 2 shows images of the form (left) of PSC before differentiation begins, a 2D single layer (middle) of mature hepatocytes, and 3D liver organoids (right), and arrows indicate 3D organoids floating on 2D cells.

FIG. 3 shows the generated 3D liver organoid (left) and an enlarged image (right) thereof.

FIG. 4 is a schematic diagram illustrating a protocol optimization process for further differentiation of liver organoids produced in an HM medium.

FIG. 5 shows results of measuring expressions of ALB and CYP3A4 in each differentiation condition. * p<0.05 and ** p<0.001.

FIG. 6 shows images showing the form of organoids cultured in suspension or Matrigel.

FIG. 7 shows the accumulated number of cells calculated in each subculturing process of liver organoids produced in an HM medium. The data are shown as an average±SEM (n=3).

FIG. 8 shows immunofluorescence images dyed with each labeled antibody in order to identify the expandability and characteristics of liver organoids produced in an HM medium.

FIG. 9 shows a result of measuring mRNA expression levels of each cell-specific genetic marker in iPSCs, hepatic endoderm differentiated cells (HE), 2D mature hepatocytes (MH), and organoids. The data are shown as an average±SEM (n=3) and analyzed by Student's t-test. * p<0.05; ** p<0.01; and *** p<0.001.

FIG. 10 shows immunofluorescence images dyed with each labeled antibody in order to analyze the characteristics of liver organoids produced in an HM medium.

FIG. 11 shows a result of FACS analysis with respect to ALB of liver organoids produced in an HM medium.

FIG. 12 shows images of organoids produced in HM conditions, EM conditions, and DM conditions.

FIG. 13 shows a result of comparing mRNA expression levels of specific markers in organoids produced in HM conditions, EM conditions, and DM conditions with primary human hepatocytes (PHH) and human hepatic tissues. The data are shown as an average±SEM (n=3) and analyzed by Student's t-test. * p<0.05; ** p<0.01; and *** p<0.001.

FIG. 14 shows immunofluorescence images of organoids in EM condition (top) and DM condition (bottom), which are dyed with each labeled antibody.

FIG. 15 shows a result of FACS analysis with respect to ALB of organoids produced in EM conditions and DM conditions.

FIG. 16 shows images in which the organoids produced in HM conditions and DM conditions are dyed with periodic acid-schiff (PAS).

FIG. 17 shows images after the organoids produced in HM conditions and DM conditions are cultured with indocyanine green (ICG) for 15 minutes.

FIG. 18 shows a result of quantifying and comparing secreted amounts of albumin (ALB) and al-antitrypsin (AAT) and urea production amounts in 2D differentiated MH, organoids produced in HM conditions and organoids produced in DM conditions, and PHH. The quantification is calculated in an amount per mL culture medium per million cells over 24 hours. The data are shown as an average±SEM (n=3) and analyzed by Student's t-test. * p<0.05; ** p<0.01; and *** p<0.001. The numerical values of human hepatic tissue-derived organoids reported by the Clever's group are indicated by a horizontal bar.

FIG. 19 shows fluorescence images of bile canaliculi-like structures dyed with CDFDA in organoids produced in HM conditions and DM conditions.

FIG. 20 shows a result of comparing gene expression levels of CYP family in 2D differentiated MH and organoids in HM conditions. * p<0.05; ** p<0.01; and *** p<0.001.

FIG. 21 shows a result of comparing mRNA expression amounts of CYP3A4 in 2D differentiated MH and organoids produced in HM conditions and DM conditions with or without 10 μM nifedipine (NIF) induction. The data are shown as an average±SEM (n=3) and analyzed by Student's t-test. ** p<0.01; and *** p<0.001.

FIG. 22 shows results of comparing NIF-induced CYP3A4 enzyme activity in 2D differentiated MH, organoids produced in HM conditions and DM conditions, and PHH. The result is presented as a relative luminescence unit (RLU) per mL per million cells. The data are shown as an average±SEM (n=3) and analyzed by Student's t-test. *** p<0.001.

FIG. 23 shows results of comparing the relative levels of residual nifedipine which remains unmetabolized by 2D differentiated MH and organoids produced in HM conditions and DM conditions after treatment of nifedipine for 12 hours. The data are shown as an average±SEM (n=3) and analyzed by Student's t-test. * p<0.05; ** p<0.01; and *** p<0.001.

FIG. 24 shows a result of comparing the relative levels of 6p-hydroxytestosterone produced by being metabolized for 12 hours after 2D differentiated MH and organoids in HM conditions are treated with testosterone. The data are shown as an average±SEM (n=3).

FIG. 25 shows images of 2D differentiated MH (top) and organoids (bottom) in HM conditions after treatment of CYP3A4- and CYP1A2/2E1-mediated hepatotoxic drugs and the like for 6 days.

FIG. 26 shows a result of measuring a toxicity concentration (TC50) with the number of cells after 2D differentiated MH and organoids in HM conditions are treated with each drug for 6 days. The data are shown as an average±SEM (n=3).

FIG. 27 shows a result of measuring the number of cells after 2D differentiated MH and organoids in HM conditions are treated with each concentration of trovafloxacin for 6 days. The data are shown as an average±SEM (n=3) and analyzed by Student's t-test. ** p<0.01; and *** p<0.001.

FIG. 28 shows a result of measuring the number of cells after 2D differentiated MH and organoids in HM conditions are treated with each concentration of trovafloxacin or levofloxacin for 6 days. The data are shown as average±SEM (n=3) and analyzed by Student's t-test. ** p<0.01; and *** p<0.001.

FIG. 29 shows an OCR result measured after treatment with 0.8 μM of trovafloxacin or levofloxacin for 6 days. The data are shown as an average±SEM (n=5) and analyzed by Student's t-test. * p<0.05.

FIG. 30 shows an OCR result measured after treatment with 4 μM trovafloxacin or levofloxacin for 6 days. The data is an average±SEM (n=5) and analyzed by Student's t-test. * p<0.05.

FIG. 31 is a schematic diagram showing an experimental process for confirming a recovery function due to toxic damage in liver organoids according to an embodiment of the present invention.

FIG. 32 shows images in which the shapes in a control group, organoids (APAP) treated with APAP for 7 days, and organoids (Recover), in which the medium is replaced after treatment with APAP for 60 hours, are observed on day 2, day 4, and day 7.

FIG. 33 shows a result of measuring and comparing sizes in a control group, organoids (APAP) treated with APAP for 7 days, and organoids (Recover) in which the medium is replaced after treatment with APAP for 60 hours. The data are shown as an average±SEM (n=20) and analyzed by Student's t-test. * p<0.05 and ** p<0.01.

FIG. 34 shows fluorescence images of organoids dyed with dihydroethidium for ROS detection and immunofluorescence images of organoids dyed with each labeled antibody in a control group, organoids (APAP) treated with APAP for 7 days, and organoids (Recover) in which the medium is replaced after treatment with APAP for 60 hours.

FIG. 35 shows a result of measuring and comparing ATP contents under each condition indicated in FIG. 31. The data are shown as an average±SEM (n=3) and analyzed by Student's t-test. ** p<0.01 and *** p<0.001.

FIG. 36 shows a result of measuring and comparing GHS/GSSG ratio under each condition indicated in FIG. 31. The data are shown as an average±SEM (n=3) and analyzed by Student's t-test. ** p<0.01 and *** p<0.001.

FIG. 37 shows a result of measuring the mRNA expression level of a gene associated with an inflammatory reaction on each indicated date in organoids (APAP injury) treated with APAP for 7 days, and organoids (APAP recover) in which the medium is replaced after treatment with APAP for 60 hours. The data are shown as an average±SEM (n=3) and analyzed by Student's t-test. * p<0.05; ** p<0.01; and *** p<0.001.

FIG. 38 shows images (top panel) showing the form of organoids in HM conditions treated with BSA, FA (oleate and palmitate), FA+etomoxir (CPT1 inhibitor), FA+L-carnitine, and FA+metformin, respectively, enlarged images (middle panel) of lipid droplets (square marked portion), and confocal fluorescence images (bottom panel) dyed with Nile red.

FIG. 39 shows a result of measuring relative Nile red intensity after the organoids in HM conditions treated with BSA, FA (oleate and palmitate), FA+etomoxir (CPT1 inhibitor), FA+L-carnitine, and FA+metformin, respectively, are dyed with Nile red. The data are shown as an average±SEM (n=3) and analyzed by Student's t-test. * p<0.05.

FIG. 40 shows a result of measuring the triglyceride concentration in the organoids in HM conditions treated with BSA, FA (oleate and palmitate), FA+etomoxir (CPT1 inhibitor), FA+L-carnitine, and FA+metformin, respectively. The data are shown as an average±SEM (n=3) and analyzed by Student's t-test. ** p<0.01 and *** p<0.001.

FIG. 41 shows a result of measuring OCR in organoids in HM conditions treated with BSA, FA (oleate and palmitate), FA+etomoxir (CPT1 inhibitor), and FA+L-carnitine, respectively. The data are shown as an average±SEM (n=5) and analyzed by Student's t-test. * p<0.05 and ** p<0.001.

FIG. 42 shows a result of screening drugs for suppressing lipid accumulation from an autophagy library in order to screen therapeutic agents for fatty liver, and shows images (top) showing the forms of liver organoids respectively treated with four drugs (Everolimus, Scriptaid, Tacedinaline and KU-0063794) which have the best effect on steatosis-induced liver organoids, and confocal images (bottom) dyed with Nile red.

FIG. 43 shows a result of comparing mRNA expression amounts of CD36, SREBP, and CPT1 in the steatosis-induced liver organoids treated with Everolimus, Scriptaid, Tacedinaline, and KU-0063794, respectively. The data are shown as an average±SEM (n=3) and analyzed by Student's t-test. * p<0.05; ** p<0.01; and *** p<0.001.

FIG. 44 shows a result of measuring triglyceride concentration in steatosis-induced liver organoids treated with Everolimus, Scriptaid, Tacedinaline, and KU-0063794, respectively. The data are shown as an average±SEM (n=3) and analyzed by Student's t-test. * p<0.05; ** p<0.01; and *** p<0.001.

FIG. 45 shows representative images of 2D MH (a condition) differentiated from PSC according to a conventional protocol, and organoids generated by 3D culturing hepatic endoderm cells differentiated from PSC in MH medium (b condition), HM medium (c condition), EM medium (d condition), or DM medium (e condition).

FIG. 46 shows a result of comparing sizes of the organoids produced in each condition. * p<0.05; ** p<0.01; and *** p<0.001.

FIG. 47 shows a result of comparing the number of the organoids produced in each condition. * p<0.05; ** p<0.01; and *** p<0.001.

FIG. 48 shows the possible number of subculturing the organoids produced in each condition.

FIG. 49 shows representative images of the organoids generated in each condition after a single subcultures (p1).

FIG. 50 shows a result of comparing the expression amounts of hepatocyte-specific markers (ALB and HNF4A) and hepatic precursor-specific markers (AFP and CK19) of the organoids produced in each condition. * p<0.05 and ** p<0.001.

FIG. 51 shows representative images of the organoids generated in each condition after twice subcultures (p2).

FIG. 52 shows a result of comparing the ALB expression amounts of the organoids generated in each condition after twice subcultures (p2) and three times subcultures (p3). * p<0.05 and ** p<0.001.

FIG. 53 shows a schematic diagram showing a process of sequentially culturing the organoids generated in HM condition (c condition) and EM condition (d condition) in EM+BMP7 and DM for further differentiation of liver organoids, and representative images of organoids differentiated by the process.

FIG. 54 shows a result of comparing ALB and CYP3A4 expression amounts of further differentiated organoids in each condition.

FIG. 55 shows representative images of liver organoids produced by culturing hepatic endoderm cells differentiated from PSCs in an HM medium, for each of subcultures.

FIG. 56 shows an image after thawing and viability before freezing and after thawing the liver organoids produced by culturing hepatic endoderm cells differentiated from PSCs in an HM medium.

FIG. 57 shows a result of analyzing karyotype after performing 40 subcultures (p40) and 50 subcultures (p50) of the liver organoids produced by culturing hepatic endoderm cells differentiated from PSCs in an HM medium.

FIG. 58 shows a result of confirming the expression amounts of hepatocyte-specific markers (ALB) and hepatic precursor-specific markers (AFP) for each of subcultures of the liver organoids produced by culturing hepatic endoderm cells differentiated from PSCs in an HM medium.

FIG. 59 shows representative images of liver organoids generated in day 3 (top) and day 9 (bottom) when each of bFGF, Oncostatin M (OSM), and ITS, or a combination thereof is removed during a process of producing liver organoids.

FIG. 60 shows a result of comparing the number of organoids generated on day 3 or day 9 for each condition in which each of bFGF, OSM, and ITS or a combination thereof is removed during a process of producing liver organoids.

FIG. 61 shows a result of comparing sizes of organoids generated on day 9 for each condition in which each of bFGF, OSM, and ITS, or a combination thereof is removed during a process of producing liver organoids.

FIG. 62 shows a result of comparing the number of cells accumulated for each condition in which each of bFGF, OSM, and ITS, or a combination thereof is removed during a process of culturing liver organoids subjected to late subcultures (p40 to p45).

FIG. 63 shows a result of confirming genetic biomarkers, among 93 adult hepatic tissue-specifically expressed genes of a liver specific gene expression panel (Lage), expressed in hepatocytes (MH) generated by 2D-culturing in MH medium, the liver organoids (HM) produced in an HM medium, liver organoids (EM) in which the liver organoids produced in the HM medium are cultured in EM medium, or liver organoids (DM) generated by culturing, in EM, the liver organoids produced in the HM medium, and then culturing in DM.

FIG. 64 shows a result of measuring the similarity between the liver organoids generated in each condition and the liver tissue.

### BEST MODE FOR CARRYING OUT THE INVENTION

Hereinafter, the present invention will be described in detail.

Medium Composition for Differentiation of Liver Organoids

In an aspect, the present invention relates to a medium composition for differentiation of liver organoids, wherein the medium composition comprises a basic fibroblast growth factor (bFGF), oncostatin M (OSM), and insulin-transferrin-selenium (ITS).

As used herein, the term “basic fibroblast growth factor (bFGF)” refers to a substance, which is known to have a function of promoting proliferation or inducing differentiation of various cells, and binds to a basic fibroblast growth factor receptor on the surface of cell, thereby exhibiting activity.

As used herein, the term “oncostatin M” refers to a protein secreted when a human macrophage cell line is stimulated with phorbol 12-mystristate 13-acetate (PMA), and is a cytokine that plays an important role in a hematopoietic process, an immune response, a metabolic process, etc.

As used herein, the term “insulin-transferrin-selenium (ITS)” refers to a substance, which is used as an additive for in vitro culture of embryos and stem cells of various mammalian species. Insulin is a polypeptide hormone that promotes absorption of glucose and amino acids, and may exhibit mitogenic effects. According to in vitro studies on preimplantation embryos in multiple mammalian species, oviduct and uterus include growth factors that stimulate cell proliferation and differentiation of the preimplantation embryos. Insulin and insulin-like growth factors play an important role in the growth and metabolism of the embryos. Transferrin is an iron-carrying protein and is also a detoxifying protein that removes metals from the medium. Iron is an essential trace element, but may exhibit toxicity in a free form. In order to supply nutrition to cells during culture, iron should be provided by binding to the transferrin in serum. Selenium (Se) is an essential trace element for various physiological functions, and is generally added to a culture medium in the form of sodium selenite, thereby serving to protect cells from oxidative damage by reducing free radical production and inhibiting lipid peroxidation.

As used herein, the term “organoid” is also referred to as an organ analog, and refers to a three-dimensional cell aggregate formed from adult stem cells (ASCs), embryonic stem cells, and induced pluripotent stem cells (iPSCs) through self-regeneration and self-organization. An organoid is an ex vivo three-dimensional organ having a small and simplified form that imitates an anatomy of an actual tissue, and enables disease modeling based on genetic information of a patient, drug screening through repeated tests, and the like by constructing the organoid from the tissue of the patient.

As used herein, the term “for differentiation of liver organoids” refers to the use for which starting cells, such as stem cells, hepatic endoderm cells, and hepatocytes, differentiate or proliferate to produce liver organoids. The production of the liver organoids encompasses all activities capable of producing and maintaining the liver organoids, such as proliferation, survival, and differentiation of the liver organoids.

As used herein, the term “medium” refers to a medium capable of supporting the proliferation, survival, and differentiation of liver organoids in vitro, and encompasses all conventional media suitable for culturing and differentiating liver organoids used in the art. The kind of the medium and culture conditions may be appropriately selected according to the kind of cells.

Specifically, the medium may generally include a cell culture minimum medium (CCMM) containing a carbon source, a nitrogen source, and trace element components. The cell culture minimum medium may include, for example, Dulbecco's Modified Eagle's Medium (DMEM), F-10, F-12, DMEM/F12, Advanced DMEM/F12, α-Minimal Essential Medium (α-MEM), Iscove's Modified Dulbecco's Medium (IMDM), Basal Medium Eagle (BME), RPMI1640, and the like, but is not limited thereto.

The medium may include an antibiotic such as penicillin, streptomycin, gentamicin, or a mixture of two or more thereof.

In one embodiment of the present invention, the Advanced DMEM/F-12 medium may be used as a basic medium for differentiation and culture of liver organoids.

In addition, the medium composition may further include at least one selected from the group consisting of PS, GlutaMAX, HEPES, N2 supplement, N-acetylcysteine, [Leu15]-Gastrin I, an epidermal growth factor (EGF), hepatocyte growth factor (HGF), vitamin A-free B27 supplement, A83-01, nicotinamide, forskolin, dexamethasone, and a combination thereof.

Meanwhile, expansion medium (EM) and differentiation medium (DM) for proliferating and differentiating hepatocytes separated from adult liver tissues into 3D-type liver organoids are well-known (see Broutier L, et al. Culture and establishment of self-renewing human and mouse adult liver and pancreas 3D organoids and their genetic manipulation. Nat Protoc 2016; 11:1724-1743).

The present inventors excluded R-spondin, which is an expensive medium additive, in the EM medium, added bFGF instead of the fibroblast growth factor 10 (FGF 10), and further added oncostatin M (OSM), insulin-transferrin-selenium (ITS), and dexamethasone thereto to form a hepatic medium (HM) (Table 1).

In an embodiment of the present invention, hepatic endoderm cells differentiated from stem cells were respectively 3D-cultured in the HM medium, the EM medium, and the DM medium to produce liver organoids, and the liver organoids were subcultured. As a result, the liver organoids produced in the DM medium were not able to be subcultured three times or more, but the liver organoids produced in the HM medium were able to be subcultured 90 times or more. In addition, it was confirmed that the liver organoids produced in the HM medium maintained the karyotype thereof and the characteristics of mature hepatocytes even after multiple subcultures.

That is, the medium for differentiating liver organoids according to the present invention can significantly increase in the ability of proliferating and differentiating liver organoids without expensive R-spondin, as compared with known EM medium.

In another embodiment of the present invention, as a result of confirming the effect of bFGF, OSM, and ITS, which are components distinguished from the previously known liver organoid culture medium (MH medium, EM medium, and DM medium), on the liver organoid generation process, the number of liver organoids generated when all three components are present may increase, and the cell proliferation ability may be the highest even in the subculture process.

Method for Producing Liver Organoids

Another aspect of the present invention relates to a method for producing liver organoids, wherein the method comprises culturing, in the medium composition, hepatic endoderm cells, or hepatocytes which are differentiated from stem cells.

The medium composition is the same as described above.

As used herein, the term “stem cells” refers to cells having the ability to differentiate into various cells through a suitable environment and stimulation, and the ability to self-proliferate, and may be adult stem cells, induced pluripotent stem cells, or embryonic stem cells.

In one embodiment of the present invention, the stem cells may be human induced pluripotent stem cells or human embryonic stem cells.

Specifically, the human induced pluripotent stem cells may be produced by reprogramming human foreskin fibroblasts or human liver fibroblasts, and the human embryonic stem cells may be an H1 cell line or an H9 cell line.

In the present invention, the culturing in the medium composition may include three dimensionally (3D) culturing the hepatic endoderm cells or the hepatocytes to differentiate into liver organoids.

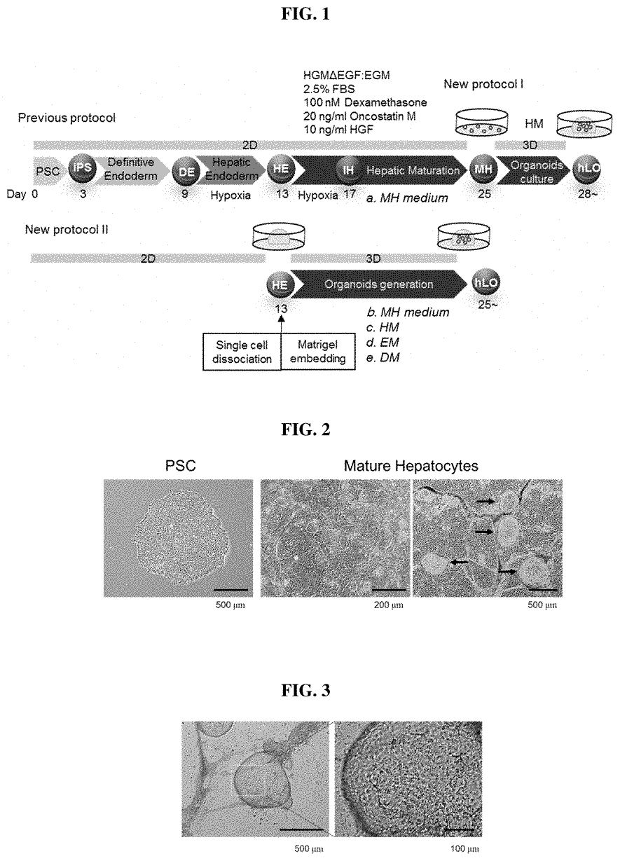

Meanwhile, in the present invention, the expandable liver organoids may be produced by modifying a previously known protocol for obtaining hepatocytes from stem cells, and gradually differentiating PSCs into definitive endoderm (DE), hepatic endoderm (HE), immature hepatocytes (IH), and mature hepatocytes (MH). In this case, the liver organoids according to the present invention may be produced by the method in that the differentiation to the mature hepatocytes may be performed by a 2D culture process, and when 3D-type liver organoids are generated on a 2D single layer in the process of the differentiation into the mature hepatocytes, the liver organoids are collected and 3D-cultured in the HM medium (Table 1) (see New protocol I of FIG. 1).

In addition, another embodiment of the present invention may include culturing the liver organoids in the EM medium supplemented with BMP7 and then sequentially culturing the liver organoids in the DM medium. The liver organoids produced through sequential culture in the EM medium and DM medium may exhibit the characteristics of more mature hepatocytes.

Meanwhile, it was confirmed that the liver organoids produced by the method of New protocol I of FIG. 1 were expandable and exhibited the characteristics of mature hepatocytes, but the process of collecting the 3D type liver organoids generated on the 2D single layer may be inconvenient, and the liver organoid generation efficiency may vary with differentiation conditions. In addition, components constituting Hepatocyte Culture Medium (Lonza; CC-3198), which is a medium used in the hepatocyte differentiation process, are not clearly known. Accordingly, the present inventors have developed a method for allowing liver organoids to be mass-produced on a medium in which components are clearly defined in a simpler manner.

The liver organoids of the present invention may be obtained by a method which is simpler and has a superior yield compared to the method of New protocol I of FIG. 1.

In another embodiment of the present invention, PSCs are differentiated into hepatic endoderm cells, and then liver organoids can be produced directly from the differentiated hepatic endoderm cells. Specifically, a process of differentiating the PSCs into the hepatic endoderm cells may be performed using a previously known method, and the differentiated hepatic endoderm cells may be isolated into single cells, may be embedded in Matrigel and solidified, and then may be 3D-cultured in the HM medium to generate the liver organoids (see New protocol II of FIG. 1).

When the liver organoids are obtained through the above method, the liver organoids can be obtained in high yield in a clearly defined medium without the inconvenient process of collecting the 3D-type liver organoids generated on the 2D single layer.

In addition, another embodiment of the present invention may include culturing the liver organoids in the EM medium supplemented with BMP7 and then sequentially culturing the liver organoids in the DM medium.

Liver Organoids

In an aspect, the present invention relates to liver organoids produced by the production method.

In another aspect, the present invention relates to liver organoids that express, as liver-specific genetic markers, AMBP, APOA2, APOB, CYP8B1, F2, FGA, FGB, FGG, HABP2, ITIH2, PROC, SERPINA11, SERPINA4, SLC2A2, UGT2B15, and VTN.

The present inventors have evaluated the differentiation state of the liver model based on RNA sequencing and constructed a liver-specific gene expression panel (LiGEP) containing 93 genes, which may exhibit liver similarity (see Kim D S, et al. a liver-specific gene expression panel predicts the differentiation status of in vitro hepatocyte models. Hepatology 2017; 66:1662-1674). Further, the present inventors have made efforts to develop liver organoids, and thus produced liver organoids which can proliferate even after 60 times or more subcultures and maintain the characteristics of mature hepatocytes, and confirmed liver-specific markers commonly expressed in the liver organoids.

In addition, the liver organoids may further express one or more liver-specific genetic markers selected from the group consisting of SLC2A2, CYP2C9, CYP2C8, UGT2B10, AKR1C4, SLC38A4, CXCL2, TAT, SLCO1B1, BAAT, F12, CPB2, SERPINA6, GC, CFHR3, APCS, SLC10A1, CXCL2, SLC38A4, and AFM.

For example, the liver organoids may express AMBP, APOA2, APOB, CYP8B1, F2, FGA, FGB, FGG, HABP2, ITIH2, PROC, SERPINA11, SERPINA4, SLC2A2, UGT2B15, VTN, CYP2C9, CYP2C8, UGT2B10, AKR1C4, SLC38A4, CXCL2, TAT, SLCO1B1, BAAT, HABP2, F12, CPB2, SERPINA6, GC, CFHR3, and APCS.

In addition, the liver organoids may express AMBP, APOA2, APOB, CYP8B1, F2, FGA, FGB, FGG, HABP2, ITIH2, PROC, SERPINA11, SERPINA4, SLC2A2, UGT2B15, VTN, SLC10A1, and CXCL2.

In addition, the hepatic organoids may express AMBP, APOA2, APOB, CYP8B1, F2, FGA, FGB, FGG, HABP2, ITIH2, PROC, SERPINA11, SERPINA4, SLC2A2, UGT2B15, VTN, SLC38A4, and AFM.

In one embodiment of the present invention, in the evaluation of the expression amount and functionality of the hepatocyte-specific gene, the liver organoids may exhibit the characteristics of more mature hepatocytes as compared with hepatic endoderm cells and hepatocytes produced by 2D-culturing pluripotent stem cells.

In another embodiment of the present invention, the liver organoids may maintain the shape thereof even after the freezing and thawing processes, and exhibit high viability.

In addition, the liver organoids can be subcultured 90 times or more.

In addition, in another embodiment of the present invention, the liver organoids can proliferate even after 90 times or more subcultures, maintain the normal karyotype, and maintain the characteristics and function as mature hepatocytes. That is, the liver organoids are expandable organoids.

In the present invention, the liver organoids can be subcultured 10 times to 100 times, 20 times to 95 times, 30 times to 90 times, 40 times to 85 times, 50 times to 80 times, 55 times to 75 times, or 60 times to 70 times.

In addition, the liver organoids may be differentiated from stem cells.

Meanwhile, the present inventors have constructed an algorithm to quantitatively and numerically express information on the similarity with liver tissue or the differentiation level into liver tissue (see Korean Patent No. 10-1920795).

In the present invention, when the expression amount of the liver tissue-specific gene measured in the liver organoids is applied to Equation 1 below, the similarity with liver tissue may be 40%, 45%, 50%, 60%, 70%, 75%, 80%, 85%, 90%, or 95% or more:

$${D}_{i} = ( 1 - \frac{{B}_{i} + {C}_{i}}{{A}_{i} + {B}_{i} + {C}_{i}} ) \times 100 ( \% ) \qquad [ Equation 1 ]$$

In Equation 1 above, Aᵢ, Bᵢ, and Cᵢ are I(yᵢ−Uᵢ>0)·I(zᵢ>0), I(yᵢ−Uᵢ≤0)·I(zᵢ>0), and I(yᵢ−Uᵢ>0)·I(zᵢ≤0), respectively; yᵢ is the value of fragments per kilobase of exon model per million mapped reads (FPKM) of the i-th gene of the liver tissue sample; Uᵢ is the upper limit value of 100×(1−α)% confidence interval of the Wilcoxon signed-rank test; and zᵢ is the difference (uᵢ−Uᵢ) between the FPKM value (uᵢ) of the liver organoids and Uᵢ.

As used herein, the term “fragments per kilobase of exon model per million mapped reads (FPKM)”, a unit used for the analysis of the results of RNA-Seq, is calculated by the following equation in order to represent the expression level of a gene:

FPKM=(fragment×10⁹)/(total mapped reads×sum of exon length)

In one embodiment of the present invention, the liver tissue-specific gene may be 93 genes shown in Table 12.

The liver tissue-specific gene may be a gene which exhibits the expression amount in the liver tissue at least twice as high as that in other tissues other than the liver tissue.

In addition, the measurement of the expression amount of the liver tissue-specific gene may be the measurement of the mRNA expression level of the gene or the expression level of the protein encoded by the gene.

Measuring the mRNA expression level of the target tissue-specific gene may be performed using an antisense oligonucleotide, a primer pair, a probe, or a combination thereof, which specifically binds to the mRNA of the gene, and may be performed using an assay selected from the group consisting of a reverse transcriptase polymerase reaction, a competitive reverse transcriptase polymerase reaction, a real-time reverse transcriptase polymerase reaction, an RNase protection assay, northern blotting, and a DNA microarray chip, but is not limited thereto.

Measuring the expression level of the protein encoded by the gene may be performed using an antibody, aptamer, or a combination thereof that specifically binds to the protein, and may be performed using an assay selected from the group consisting of western blotting, ELISA, radioimmunoassay, radioimmunodiffusion, Ouchterlony immunodiffusion, rocket immunoelectrophoresis, immunohistochemical staining, immunoprecipitation assay, complement fixation assay, FACS, and protein chips, but is not limited thereto.

Method for Screening Hepatotoxic Drugs

In another aspect, the present invention relates to a method for screening hepatotoxic drugs, wherein the method comprises: contacting a test substance with the liver organoids; and measuring cell viability or an oxygen consumption rate (OCR) in the liver organoids.

As used herein, the term “test substance” may be an individual nucleic acid, protein, other extract or natural substance, a compound, or the like which is presumed to have the possibility of preventing or treating liver-related diseases or is randomly selected according to a conventional selection method.

In the present invention, the method for screening hepatotoxic drugs may be carried out by treating the liver organoids with a test substance to measure the cell viability or oxygen consumption rate, and then comparing the measured values with that of a control group which is not treated with the test substance.

Specifically, the method may be carried out in a way that, in the case of treating the liver organoids with the test substance, when the cell viability decreases or the oxygen consumption rate decreases, the test substance may be determined as a hepatotoxic substance. The oxygen consumption rate is for determining the functionality of the mitochondria, and it may be confirmed that the respiration of the mitochondria is decreased through the reduction of the oxygen consumption rate.

In one embodiment of the present invention, it may be confirmed that the liver organoids are remarkably high in the sensitivity and accuracy to the toxic drug as a result of comparing the cell viability and oxygen consumption rate with those of 2D differentiated MH.

Method for Screening Therapeutic Agents for Fatty Liver

In still another aspect, the present invention relates to a method for screening therapeutic agents for fatty liver, wherein the method comprises: producing the liver organoids into fatty liver organoids; and treating the fatty liver organoids with candidate materials for therapeutic agents for fatty liver.

In the present invention, the producing of the liver organoids into fatty liver organoids may include administering a fatty acid to the liver organoids.

The fatty acid may be oleate, palmitate, or a mixture thereof, but is not limited thereto.

In the present invention, when the liver organoids are treated with the test substance, the method for screening the therapeutic agents for fatty liver may be carried out in a way that the candidate material is determined as a therapeutic agent for fatty acid when i) the expression of genes and proteins involved in glucogenesis and lipogenesis is significantly reduced, ii) the stain of triglyceride and the amount of triglyceride in cells are significantly reduced, or iii) the ability to absorb glucose is increased, as compared with a control group that is not treated with the test substance.

In one embodiment of the present invention, when the liver organoids are treated with oleate and palmitate, the concentration of triglyceride increases and OCR decreases, and thus it may be confirmed that hepatic steatosis is induced.

The substance selected by means of such a screening method acts as a leading compound in the subsequent course of developing an agent for preventing or treating fatty liver, and a new agent for preventing or treating fatty liver can be developed by transforming and optimizing the leading compound.

### MODE FOR THE INVENTION

Hereinafter, the present invention will be described in more detail with reference to Examples. The following Examples are intended to only illustrate the present invention without limiting its scope.

I. Preparation of Hepatic Endoderm Cells from Pluripotent Stem Cells (PSCs)

### Example 1. Preparation of Pluripotent Stem Cells

### Example 1.1. Preparation of Human Foreskin Fibroblast-Derived iPSCs

Human foreskin fibroblasts (HFFs) (CRL-2097) was purchased from American Type Culture Collection (ATCC). CRL-2097 HFFs were seeded on a 6-well plate at 2×10⁵ cells/well, and were transduced with Sendai virus on day 2 using CytoTune®-iPS 2.0 Sendai Reprogramming Kit (Thermo Fisher; A16517). On day 3, the medium was replaced with a fresh fibroblast culture medium (DMEM containing 10% fetal bovine serum, 1% NEAA, 1 mM L-glutamine and 0.1 mM b-mercaptoethanol), and then on day 9, the HFFs were transferred from the 6-well plate to an MEF feeder layer at 1×10⁵ cells/well. The next day, the medium was replaced with a PSC medium, and replaced with a fresh medium daily. On about day 22 of reprogramming, an iPSC colony was selected.

### Example 1.2. Preparation of Human Liver Fibroblast-Derived iPSCs

Human liver fibroblasts (HLFs) were isolated from human liver tissues, which were approved by Chungnam National University Hospital Institutional Review Board (IRB File No. CNUH 2016-03-018). Informed consent was obtained from all patients. Fresh human liver biopsy samples were washed with cold phosphate buffer saline (PBS) to remove blood, and then was treated with type 4 collagenase (300 units/ml, Thermo Fisher; 17104-019). The tissue was cut with a fine surgical blade and incubated at 37° C. for 30 minutes. Digested liver tissues were washed with cold PBS and then filtered with 70 m strainer (SPL Life Sciences; 93070). The collected cells were washed with cold PBS containing 10% fetal bovine serum (FBS, RMBIO; FBS-BBT-5XM), and then were resuspended in a minimal essential medium (MEM, Thermo Fisher; 11095-080) supplemented with preheated 10% FBS and 1% penicillin-streptomycin (PS, Thermo Fisher; 15140-122).

The HLFs were reprogrammed by using Neon Transfection System (Thermo Fisher; MPK5000). Specifically, pCXLE-hOCT4-shp53 (2.5 μg), pCXLE-hSK (2 μg), and PCXLE-hUL (2 μg) plasmid DNA cocktail were transduced by electroporation under the conditions of 1650 V, 20 milliseconds and one time pulse according to the manufacturer's instructions. After transduction, the cells were seeded to a dish coated with Matrigel™ (Corning; 354234) and incubated in a minimal essential medium (MEM, Thermo Fisher; 11095-080) supplemented with 10% FBS and 1% penicillin-streptomycin (PS, Thermo Fisher; 15140-122). The next day, the medium was replaced with mTeSR™1. On about day 22 of reprogramming, an iPSC colony was selected.

### Example 1.3. Preparation of Human Embryonic Stem Cells

Human embryonic stem cell line H1 (WiCell Research Institute, WA01) and H9 (WiCell Research Institute, WA09) were maintained at 37° C. under 5% CO₂ condition on a γ-irradiated mouse embryonic fibroblast (MEF) feeder in DMEM/F12 medium (Thermo Fisher; 11330) containing 20% knockout serum replacement (SR, Thermo Fisher; 10828-028), 1% PS, 0.1 mM 2-mercaptoethanol (Thermo Fisher; 21985-023), 1% non-essential amino acids (Thermo Fisher; 11140), 1% GlutaMax I (Thermo Fisher; 35050-079), and 10 ng/mL bFGF (PeproTech; 100-18B), or in mTeSR™1 (Stem Cell Technologies; 85850) of a Matrigel-coated plate. PSC colonies were scrapped with a 23G needle (BD bioscience; 302006) or treated with type 4 collagenase (Invitrogen; 17104-019) into small pieces, and then transferred to a new feeder weekly.

### Example 2. Preparation of Hepatic Endoderm Cells from Pluripotent Stem Cells

According to a method in which protocols described in Si-Tayeb K, et al. Hepatology 2010; 51:297-305, Takebe T, et al. Nature 2013; 499:481-484, and Takebe T, et al. Nat Protoc 2014; 9:396-409 are modified, the pluripotent stem cells (PSCs) prepared in Examples 1.1, 1.2 and 1.3 were differentiated into hepatic endoderm (HE) cells. Referring to FIG. 1, it corresponds to the differentiation process of PSC→DE→HE.

First, in order to differentiate the PSCs into definitive endoderm (DE) cells, the PSCs were cultured for 3 days in DMEM/F12 medium (Thermo Fisher; 11330) containing 20% knockout serum replacement (SR, Thermo Fisher; 10828-028), 1% PS, 0.1 mM 2-mercaptoethanol (Thermo Fisher; 21985-023), 1% non-essential amino acids (Thermo Fisher; 11140), 1% GlutaMax I (Thermo Fisher; 35050-079), and 10 ng/mL bFGF (PeproTech; 100-18B) on a Matrigel-coated plate, and then were cultured for 6 days by replacing the culture with RPMI 1640 (Thermo Fisher; 11875-093) medium supplemented with insulin-free 1×B27 (Thermo Fisher; A1895601) and 100 ng/mL human activin A (PeproTech; 120-14e).

Next, in order to differentiate the definitive endoderm cells into hepatic endoderm (HE) cells, the cells were cultured for 4 days under hypoxic conditions by replacing the medium with RPMI 1640 medium supplemented with 1×B27 (Thermo Fisher; 17504-044), 10 ng/mL bFGF, and 20 ng/mL human BMP-4 (PeproTech; 120-05ET).

II. Preparation of Liver Organoids Including Hepatic Maturation Process

### Example 3. Preparation of Liver Organoids

### Example 3.1. Differentiation into Mature Hepatocytes According to Conventional Protocol

For the differentiation from hepatic endoderm cells into hepatocytes, each medium for the hepatic endoderm cells obtained in Example 2 was replaced with a mature hepatocyte (MH) medium. The MH medium was prepared by diluting, in a ratio of 1:1, Endothelial Cell Growth Medium-2 (Lonza; CC-3162) and Hepatocyte Culture Medium (Lonza; CC-3198) supplemented with 2.5% FBS, 100 nM dexamethasone (Sigma-Aldrich; D4902), 20 ng/mL OSM (R&D system; 295-OM-050), and 10 ng/mL HGF (PeproTech; 100-39) without EGF, and the composition is listed in Table 1. The hepatic endoderm cells were cultured under hypoxic conditions for 4 days to be differentiated into immature hepatocytes (IH), and then further cultured for 8 days under normoxic conditions to be differentiated into mature hepatocytes (MH). Referring to FIG. 1, it corresponds to the differentiation process of HE→IH→MH.

### Example 3.2. Preparation of Liver Organoids Using HM Medium

After 9 days to 12 days of 2D-culturing each of the hepatic endoderm cells obtained in Example 2 in the MH medium, 3D-type liver organoids appeared on the 2D single layer of the mature hepatocytes (FIG. 2), and the cuboidal cell form similar to that of actual hepatocyte clearly appeared on the surface of a spherical structure (FIG. 3). The generated 3D-type liver organoids were collected, and then were embedded in Matrigel and solidified.

In addition, in order to enhance the self-regenerative potential of organoids and the characteristics of mature hepatocytes, R-spondin and fibroblast growth factor 10 (FGF 10) were excluded in the expansion medium (EM) of Hans Clever's group described in Broutier L, et al. Culture and establishment of self-renewing human and mouse adult liver and pancreas 3D organoids and their genetic manipulation. Nat Protoc 2016; 11:1724-1743, basic fibroblast growth factor (bFGF), oncostatin M (OSM), insulin-transferrin-selenium (ITS), and dexamethasone were further added to form a hepatic medium (HM) (Table 1). Then, the solidified organoids were 3D-cultured in the HM medium to produce liver organoids (FIG. 1).

### Example 3.3. Further Differentiation of Liver Organoids Produced in HM Medium

For further differentiation of liver organoids produced in the HM medium, the liver organoids were sequentially cultured in a differentiation medium (DM) and/or the expansion medium (EM) of Hans Clever's group described in Broutier L, et al. Culture and establishment of self-renewing human and mouse adult liver and pancreas 3D organoids and their genetic manipulation. Nat Protoc 2016; 11:1724-1743.

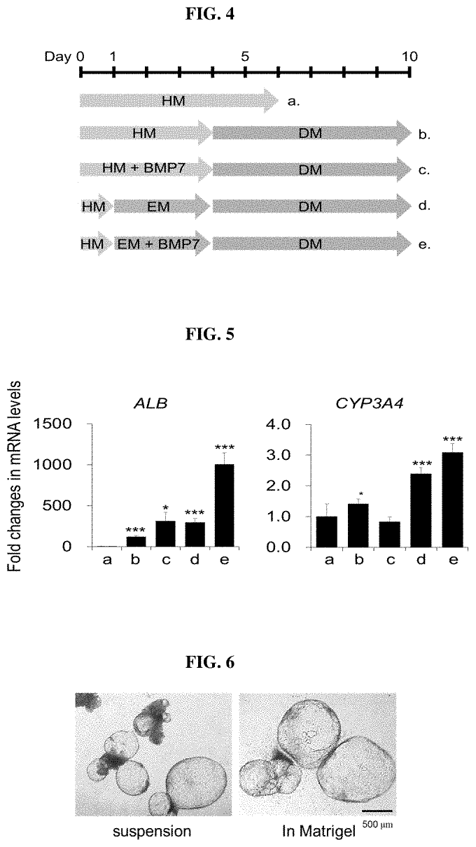

Meanwhile, for optimization of the further differentiation process, as a result of culturing the liver organoids under various conditions, it was confirmed that the addition of BMP7 to the EM medium before culturing the liver organoids in the DM medium is the most effective condition for increasing the expression of ALB, a hepatocyte-specific marker, and CYP3A4 that plays an important role in drug metabolism and toxicity (e condition in FIGS. 4 and 5). Specifically, the liver organoids were cultured in the EM medium supplemented with 20 ng/mL BMP7 (PeproTech; 120-03) for 2-3 days, and further cultured in the DM medium for 6 days.

In addition, the condition in which the liver organoids were produced in the JIM medium was designated as “HIM,” the condition in which the liver organoids produced in the HM medium were further cultured in the EM medium for 6 days was designated as “EM,” and the condition in which the liver organoids produced in the JIM medium were cultured in the EM medium together with BMP7 for 2 days, and then were cultured in the DM medium for 6 days was designated as “DM.”

The composition of each of the MH, JIM, EM, and DM media is as shown in Table 1 below:

### Experimental Example 1. Evaluation of Proliferation Ability of Liver Organoids

The liver organoids (derived from CRL-2097) obtained in Example 3.2 were typically maintained in the HM medium, and the medium was replaced every 3 days. In addition, the liver organoids were physically subcultured every 7 days. The liver organoids were washed with cold PBS to remove Matrigel, and divided into small pieces using a surgical knife under a dissecting microscope. The subcultured organoids were resuspended in Matrigel in a ratio of 1:3 to 1:10. Alternatively, the organoids were chemically subcultured by pipetting about 15 times with Gentle Cell Dissociation Reagent (Stem Cell Technology; ST07174).

As a result, the liver organoids produced in the HM medium were able to be self-regenerated in both the suspension and the Matrigel (FIG. 6).

Meanwhile, the liver organoids were isolated into single cells at 37° C. using TrypLE Express (Thermo Fisher Scientific; 12650-010) and stained with trypan blue, and then the number of cells in each subculture was counted by using Countess II Automated Cell Counter (Thermo fisher; AMQAX1000). The cumulative number of cells was calculated using the following equation: C(n)=[y(n)/y(n−1)]×C(n−1) (number of cells in the previous subculture/number of seeded cells in the previous subculture)×the cumulative number of cells in the previous subculture.

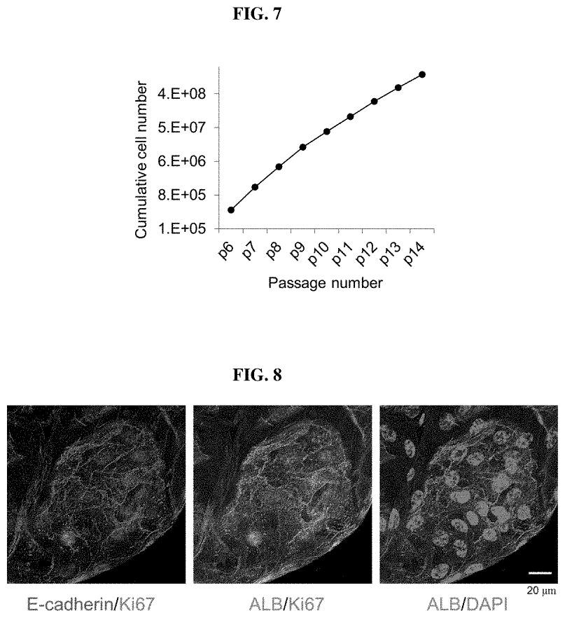

As a result, the liver organoids produced in the HM medium were able to proliferate even after multiple subcultures (FIG. 7).

In addition, in order to perform immunocytochemical analysis of the liver organoids, samples were fixed with 4% paraformaldehyde (PFA) (Biosesang; P2031) in PBS at room temperature (RT) for 15 minutes and permeabilized at 0.25% Triton X-100 at room temperature for 15 minutes. The samples were blocked with 4% bovine serum albumin (BSA)/PBS at room temperature for 1 hour, and then cultured with primary antibodies at 4° C. overnight. The samples were washed with 0.05% Tween-20 (Sigma-Aldrich; P9416) in PBS, and then cultured with secondary antibodies conjugated with Alexa Fluor® at room temperature for 1 hour. DAPI reagent (Sigma-Aldrich; D5942) was used to stain the nuclei, and fluorescence images were taken with an Olympus microscope or a Zeiss confocal microscope. The used antibodies are as listed in Table 2 below.

TABLE 2

| Antibodies | Catalog No. | Company | Dilution |
|---|---|---|---|
| anti-E-cadherin | 610181 | BD biosciences | 1:200 |
| anti-ALB | A80-129a | Bethyl lab | 1:100 |
| anti-Ki67 | Ab15580 | Abcam | 1:100 |

As a result, E-cadherin-stained epithelial cells of the liver organoids produced in the HM medium exhibited a Ki67-positive expandable state with strong expression of ALB (FIG. 8).

### Experimental Example 2. Characterization of Liver Organoids

### Experimental Example 2.1. Liver Organoids Produced in HM Medium

The characteristics of the liver organoids (derived from CRL-2097) obtained in Example 3.2 were compared with those of iPSCs obtained in Example 1, hepatic endoderm cells (HE) obtained in Example 2, and 2D differentiated mature hepatocytes (2D) MH) obtained in Example 3.1.

In order to compare gene expression levels of each cell-specific marker, RNA was extracted and quantitative real-time polymerase chain reaction (qRT-PCR) was performed. Total RNA was purified according to the manufacturer's instructions by using TRIzol reagent (Thermo Fisher; 15596018) or RNeasy Mini Kit (Qiagen; 74134). Reverse transcription was performed by using TOP Script™ RT DryMIX, dT18 plus (Ezynomics; RT200). qRT-PCR was performed by using Fast SYBR® Green Master Mix (Applied Biosystems; 4385614) as a gene-specific primer under 7500 Fast Real-Time PCR System (Applied Biosystems). The used primer sequences are as listed in Table 3 below.

TABLE 3

| Primer | Base sequence | SEQ ID NO. |
|---|---|---|
| NANOG_F | GCAGAAGGCCTCAGCACCTA | 1 |
| NANOG_R | AGGTTCCCAGTCGGGTTCA | 2 |
| LGR5_F | GACTTTAACTGGAGCACAGA | 3 |
| LGR5_R | AGCTTTATTAGGGATGGCAA | 4 |
| FOXA2_F | TGGGAGCGGTGAAGATGGAAGGGCAC | 5 |
| FOXA2_R | TCATGCCAGCGCCCACGTACGACGAC | 6 |
| SOX17_F | CGCTTTCATGGTGTGGGCTAAGGACG | 7 |
| SOX17_R | TAGTTGGGGTGGTCCTGCATGTGCTG | 8 |
| HNF1B_F | GCCCACACACCACTTACTTCG | 9 |
| HNF1B_R | GTCCGTCAGGTAAGCAGGGAC | 10 |
| HNF4A_F | GGCCAAGTACATCCCAGCTTT | 11 |
| HNF4A_R | CAGCACCAGCTCGTCAAGG | 12 |
| SOX9_F | GGAAGTCGGTGAAGAACGGG | 13 |
| SOX9_R | TGTTGGAGATGACGTCGCTG | 14 |
| CK19_F | CGCGGCGTATCCGTGTCCTC | 15 |
| CK19_R | AGCCTGTTCCGTCTCAAACTTGGT | 16 |
| ALB_F | TTTATGCCCCGGAACTCCTTT | 17 |
| ALB_R | AGTCTCTGTTTGGCAGACGAA | 18 |
| TTR_F | TGGGAGCCATTTGCCTCTG | 19 |
| TTR_R | AGCCGTGGTGGAATAGGAGTA | 20 |
| CK18_F | GAGCTGCTCCATCTGTAGGG | 21 |
| CK18_R | CACAGTCTGCTGAGGTTGGA | 22 |
| RBP4_F | GAGTTCTCCGTGGACGAGAC | 23 |
| RBP4_R | TCCAGTGGTCATCATTTCCTTTC | 24 |

Compared with the 2D MH, the liver organoids had low expression of NANOG, pluripotency marker, maintained the expression of adult stem cell marker LGR5, and expressed similar or higher levels of ductal markers SOX9 and CK19 and MH markers (ALB, TTR, CK18, and RBP4) (FIG. 9).

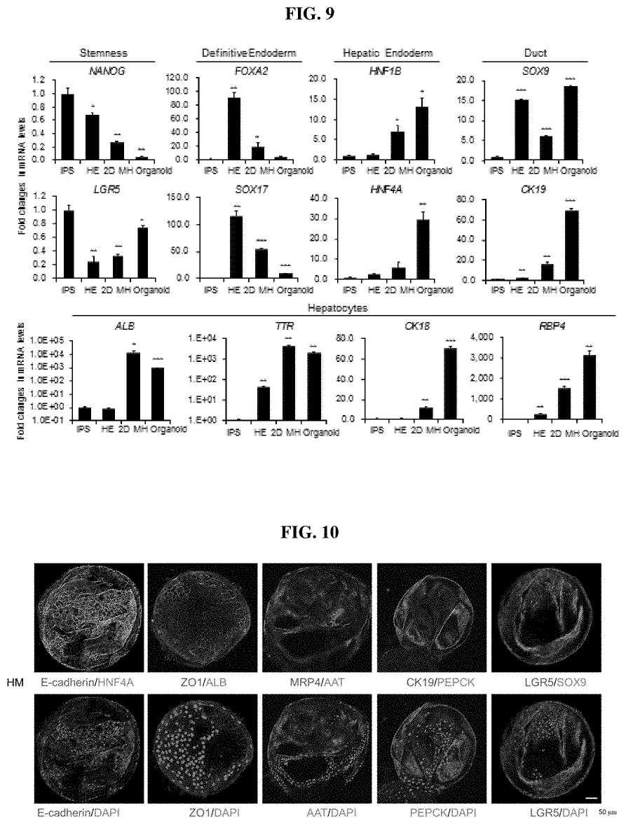

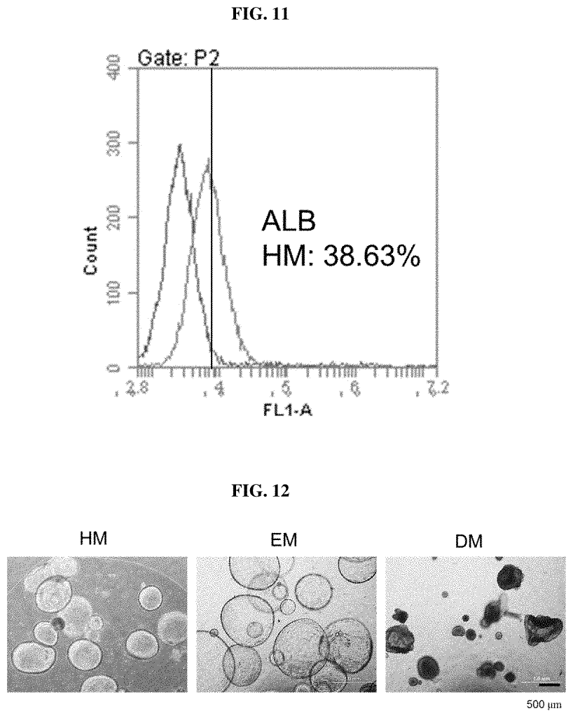

The expressions of epithelial markers (E-cadherin and ZO1), hepatocyte markers (HNF4A, ALB, AAT, and PEPCK), a bile salt efflux transporter (MRP4), ductal markers (CK19 and SOX9), and the adult stem cell marker (LGR5) were immunocytochemically analyzed at protein level according to the method described in Experimental Example 1, and as a result, the expression level was high (FIG. 10). The used antibodies are as listed in Table 4 below.

TABLE 4

| Antibodies | Catalog No. | Company | Dilution |
|---|---|---|---|
| anti-E-cadherin | 610181 | BD biosciences | 1:200 |
| anti-ALB | A80-129a | Bethyl lab | 1:100 |
| anti-ZO1 | 40-2200 | Thermo | 1:100 |
| anti-HNF4a | 3113s | Cell signaling technology | 1:200 |
| anti-MRP4 | ab15602 | abcam | 1:100 |
| anti-AAT | ab9373 | Abcam | 1:200 |
| anti-CK19 | ab9221 | Abcam | 1:300 |
| anti-PEPCK | sc-32879 | Santacruz | 1:100 |
| anti-LGR5 | TA503316 | Origene | 1:100 |
| anti-SOX9 | ab5535 | Abcam | 1:250 |
| anti-PEPCK | sc-32879 | Santacruz | 1:100 |

In addition, in order to quantify ALB+ mature hepatocyte population in the liver organoids, fluorescence activated cell sorter (FACS) analysis was performed. The liver organoids were isolated into single cells at 37° C. for 10 minutes by using TrypLE (Thermo Fisher; 12605-010) and then filtered through 30-μm mesh (Miltenyi Biotech; 130-098-458). The single cells were fixed, permeabilized, and blocked according to an immunostaining protocol. The single cells were stained with ALB-specific antibodies listed in Table 4, and then analyzed with BD Accuri™C6 (BD Biosciences).

As a result, the ALB+ mature hepatocyte population accounted for 38.63% of the liver organoids (FIG. 11).

### Experimental Example 2.2. Further Differentiated Liver Organoids

As a result of comparing the shape of the liver organoids cultured in the HM, EM, and DM conditions of Example 3.3, the organoids in the EM conditions exhibited an expanded spherical structure compared with the organoids in the HM conditions, and the organoids in the DM conditions exhibited a smaller and packed shape compared with the organoids in the HM conditions (FIG. 12).

In order to compare expression amounts of the mature hepatocyte-specific marker and the ductal marker of the organoids in each condition, qRT-PCR was performed according to the method described in Experimental Example 2.1. The used primer sequences are as listed in Table 5 below.

TABLE 5

| | | SEQ |
|---|---|---|
| Primer | Base sequence | ID NO. |
| ALB_F | TTTATGCCCCGGAACTCCTTT | 17 |
| ALB_R | AGTCTCTGTTTGGCAGACGAA | 18 |
| TTR_F | TGGGAGCCATTTGCCTCTG | 19 |
| TTR_R | AGCCGTGGTGGAATAGGAGTA | 20 |
| CK19_F | CGCGGCGTATCCGTGTCCTC | 25 |
| CK19_R | AGCCTGTTCCGTCTCAAACTTGGT | 26 |
| CYP3A4_F | CTTCATCCAATGGACTGCATAAAT | 27 |
| CYP3A4_R | TCCCAAGTATAACACTCTACACAGACAA | 28 |

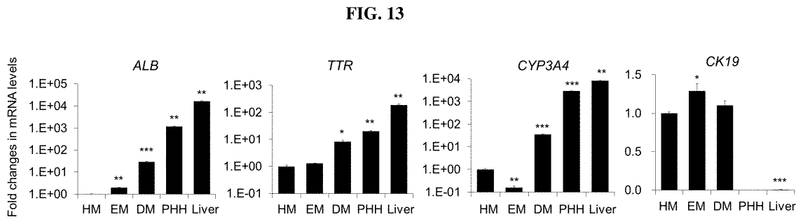

As a result, the organoids in the DM conditions expressed at significant levels the mature hepatocyte markers such as ALB, TTR and cytochrome p450-3A4 (CYP3A4) and the ductal marker CK19 as compared with PHH and human liver tissues (FIG. 13).

In addition, the expression amounts of the epithelial markers (E-cadherin and ZO1), hepatocyte markers (HNF4A, ALB, AAT, and PEPCK), bile salt efflux transporter (MRP2), ductal markers (CK19 and SOX9), and adult stem cell marker (LGR5) of the organoids cultured in the EM and DM conditions were immunochemically analyzed at protein level according to the method described in Experimental Example 1. The used antibodies are as listed in Table 6 below.

TABLE 6

| Antibodies | Catalog No. | Company | Dilution |
|---|---|---|---|
| anti-E-cadherin | 610181 | BD biosciences | 1:200 |
| anti-ALB | A80-129a | Bethyl lab | 1:100 |
| anti-ZO1 | 40-2200 | Thermo | 1:100 |
| anti-HNF4a | 3113s | Cell signaling technology | 1:200 |
| anti-MRP2 | ALX-801-016 | Enzo Life Sciences, Inc. | 1:100 |
| anti-AAT | ab9373 | Abcam | 1:200 |
| anti-CK19 | ab9221 | Abcam | 1:300 |
| anti-PEPCK | sc-32879 | Santacruz | 1:100 |
| anti-LGR5 | TA503316 | Origene | 1:100 |
| anti-SOX9 | ab5535 | Abcam | 1:250 |
| anti-PEPCK | sc-32879 | Santacruz | 1:100 |

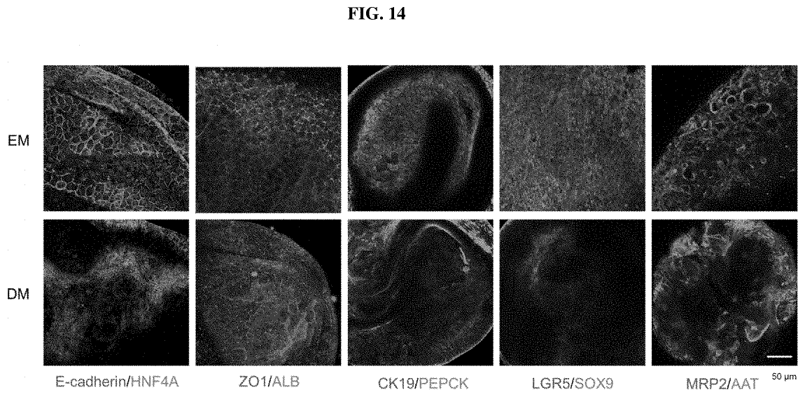

As a result, high expression levels of E-cadherin, HNF4A, ZO1, and PEPCK were shown in both conditions. Specifically, the liver organoids in the DM conditions had an increase in the expression of ALB, AAT, and MRP2, while had a decrease in the expression of CK19, LGR5, and SOX9 as compared with the liver organoids in the EM conditions (FIG. 14).

In addition, in order to quantify the ALB+ mature hepatocyte population in the liver organoids in the EM and DM conditions, FACS analysis was performed according to the method described in Experimental Example 2.1.

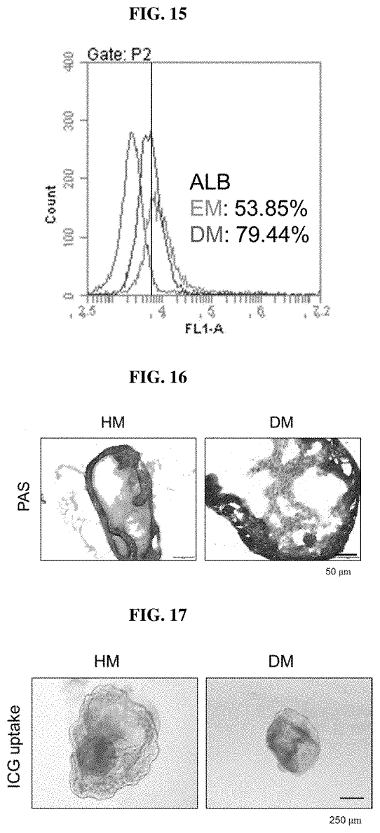

As a result, the ALB+ mature hepatocyte population accounted for 53.85% in the EM conditions and 79.44% in the DM conditions (FIG. 15).

Accordingly, it was confirmed that the organoids in the HM conditions were differentiated into more mature hepatocytes when further cultured in the DM medium.

### Experimental Example 3. Evaluation of Functionality of Liver Organoids

To analyze glycogen storage, each organoid obtained in Example 3.3 was fixed with 4% paraformaldehyde (Biosesang; P2031), cryoprotected with 30% sucrose, and frozen in an optimal-cutting-temperature (OCT) compound (Sakura Finetek; 4583). The frozen compartment was sliced to a thickness of 10 μm by using a cryostat microtome (Leica) at −20° C. The comparted samples were stained with periodic acid-schiff (PAS) (IHC World; IW-3009) according to the manufacturer's instructions.

Also, in order to analyze the absorption and release of indocyanine green (ICG), the organoids were washed with cold PBS to remove the Matrigel, and cultured with 1 mg/mL ICG (Sigma; 12633) at 37° C. under 5% CO₂ for 15 minutes. ICG absorption photos were taken with a microscope, the organoids were gently washed three times with PBS, and then a new medium was added. Then, after the culture at 37° C. under 5% CO₂ for 1 hour, ICG release photos were taken with a microscope.

As a result, ICG absorption used as a functional evaluation for PAS staining and human liver transplantation was strongly detected in the organoids in the HM and DM conditions (FIGS. 16 and 17).

In order to quantify secretion amount of ALB and AAT, and production amount of urea, the medium was collected 48 hours after the medium was replaced, and the medium was analyzed by using Human Albumin ELISA Kit (Bethyl Laboratories; E80-129), Human Alpha-1-Antitrypsin ELISA Quantitation Kit (GenWaybio; GWB-1F2730), or Urea Assay Kit (Cell Biolabs, Inc.; STA-382) according to the manufacturer's instructions. Absorbance was measured with Spectra Max M3 Microplate Reader (molecular devices) and the data was normalized to the number of cells.

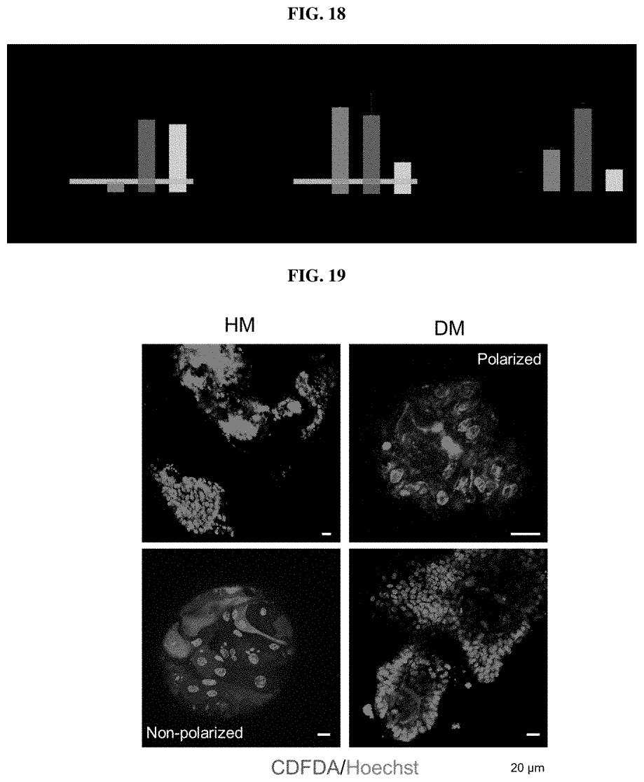

As a result of comparing the amount of ALB secretion, compared with the 2D MH or organoids cultured in HM conditions, the amount of ALB secreted in the organoids in the DM conditions was significantly increased to levels similar to that in the PHH (the left in FIG. 18). The secretion amount of AAT was significantly increased in the organoids in the HM or DM conditions compared with that in the 2D MH or PHH (the middle in FIG. 18). In addition, the production amount of urea was also significantly increased in the organoids in the HM or DM conditions (the right in FIG. 18).

Meanwhile, for functional polarization analysis, the organoids were separated from Matrigel, and cultured in a culture medium supplemented with 10 μg/mL CDFDA (Sigma; 21884) and 1 μg/mL Hoechst 33342 (Invitrogen; 62249) at 37° C. under 5% C02 for 30 minutes. The organoids were gently washed twice with cold PBS containing calcium and magnesium. After the addition of the culture medium, fluorescence images were obtained with a confocal microscope at 37° C. under 5% CO₂.

As a result, polarized epithelial cells with bile duct-like structures were clearly detected in the organoids in the HM and DM conditions by CDFDA staining (FIG. 19), which is known to be difficult to be detected in the 2D single layer culture system.

Accordingly, it was confirmed that the liver organoids cultured in the HM or DM conditions exhibit functionally mature hepatocyte-like characteristics.

### Experimental Example 4. Analysis of Drug Metabolism of Liver Organoids

To compare the gene expression levels of the CYP family including CYP3A4, 1A2, 2A6, and 2E1, which are important for drug metabolism and toxicity, qRT-PCR was performed according to the method described in Experimental Example 2.1. The used primer sequences are as listed in Table 7 below.

TABLE 7

| | | SEQ |
|---|---|---|
| Primer | Base sequence | ID NO. |
| CYP3A4_F | CTTCATCCAATGGACTGCATAAAT | 25 |
| CYP3A4_R | TCCCAAGTATAACACTCTACACAGACAA | 26 |
| CYP3A7_F | AAACTTGGCCGTGGAAACCT | 27 |
| CYP3A7_R | CAGCATAGGCTGTTGACAGTC | 28 |
| CYP1A2_F | CTTCGCTACCTGCCTAACCC | 29 |
| CYP1A2_R | GACTGTGTCAAATCCTGCTCC | 30 |
| CYP2A6_F | CAGCACTTCCTGAATGAG | 31 |
| CYP2A6_R | AGGTGACTGGGAGGACTTGAGGC | 32 |
| CYP2E1_F | TTGAAGCCTCTCGTTGACCC | 33 |
| CYP2E1_R | CGTGGTGGGATACAGCAA | 34 |

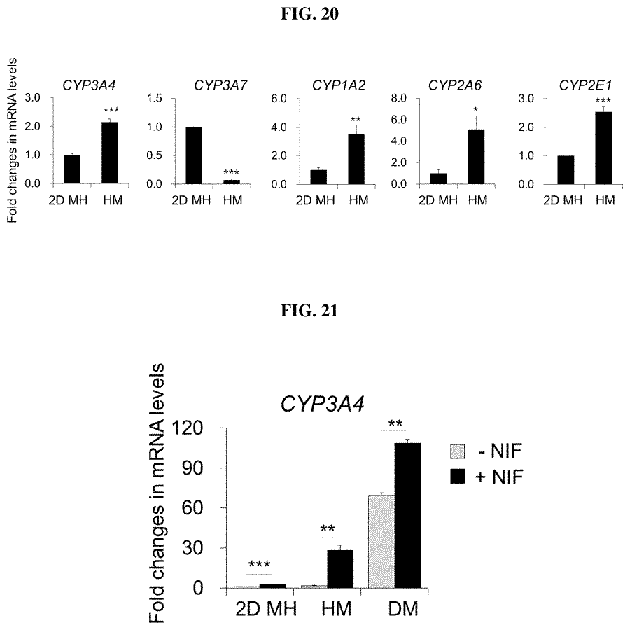

As a result, the expression amounts of CYP3A4, 1A2, 2A6, and 2E1 were significantly increased in the organoids in the HM conditions as compared with the 2D MH cultured organoids (FIG. 20). In particular, the expression of CYP3A7, a fetal gene corresponding to CYP3A4, which accounts for a major proportion of CYP-mediated drug metabolism, was significantly reduced in the organoids in the HM conditions compared with the expression in the 2D MH (FIG. 20), which means that the organoids in the HM conditions exhibits the characteristics of more mature hepatocytes.

Meanwhile, for additional study on drug metabolism, the organoids cultured in each condition were treated with 10 μM nifedipine (Sigma; N7634) for 48 hours to induce the expression of CYP3A4. Then, qRT-PCR was performed according to the method described in Experimental Example 2.1 to measure the expression amount of CYP3A4.

As a result, the expression amount of CYP3A4 was the highest in the organoids in the DM conditions compared with those of the 2D MH and the organoids in HM conditions, and significantly increased when induced by nifedipine (FIG. 21).

In addition, to measure the activity of CYP3A4, the organoids cultured in each condition were treated with 20 μM rifampicin (Sigma; R7382), 100 μM acetaminophen (APAP) (Sigma; A5000), and 10 μM nifedipine for 48 hours to induce the activity of CYP3A4. Then, the organoids were cultured with a subtype-specific matrix of CYP3A4 for 3 hours, and then the activity of CYP3A4 was measured by using P450-Glo Assay Kit (Promega; V9002 for 3A4 and V8422 for 1A2). The data was normalized to the number of cells.

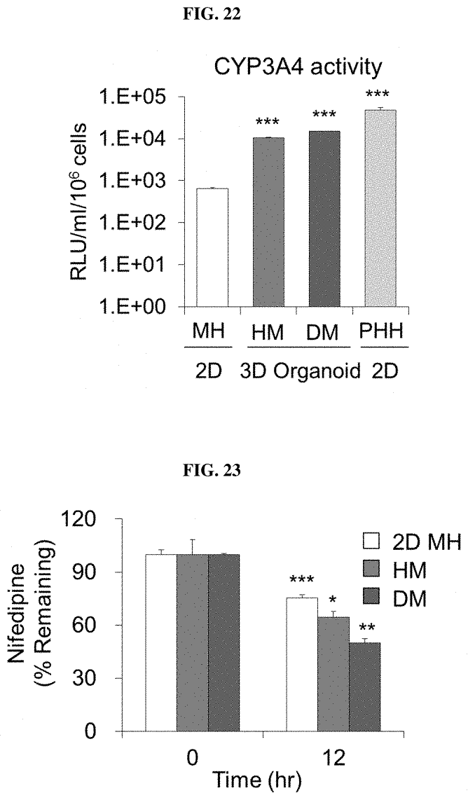

As a result, in the case of induction with nifedipine, CYP3A4 activity similar to PHH was exhibited in the organoids in both the HM and DM conditions (FIG. 22).

Meanwhile, the 2D differentiated mature hepatocytes, and the organoids in the HM and DM conditions were induced with 10 μM nifedipine for 48 hours, followed by the addition of nifedipine or testosterone. After treatment, 100 μL of supernatant was obtained at a designated time point, and the remaining nifedipine or generated 6p-hydroxytestosterone was measured with a liquid chromatography electrospray ionization tandem mass spectrometry (4000 QTRAP LC-MS/MS system equipped with LC-ESI/MS/MS, Agilent 1200 HPLC and Turbo V™ Ion Spray source). Aliquots (100 μL) were diluted with 2-fold volume of acetonitrile containing carbamazepine as an internal standard for sample analysis and transferred to a 96-well plate.

As a result, the residual amount of nifedipine remaining in the supernatant was reduced in the organoids in the HM or DM conditions compared with the organoids cultured in the 2D MH (FIG. 23), which means that the detoxification function of the 3D-cultured organoids is superior to the 2D-cultured organoids.

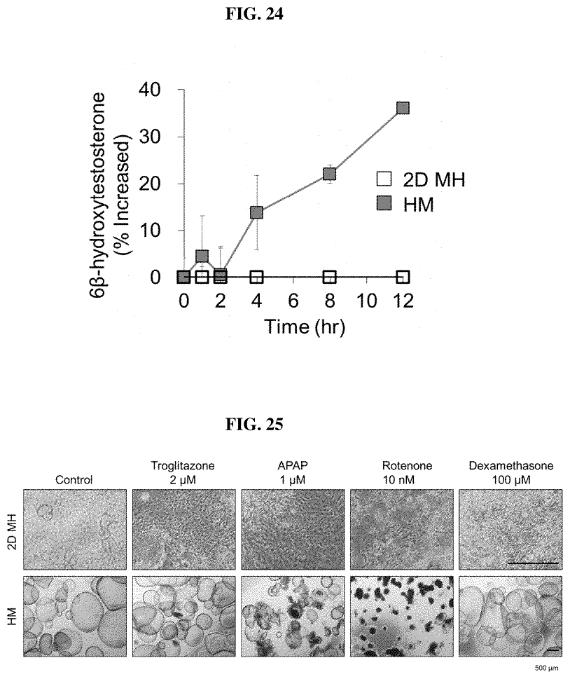

Furthermore, the organoids in the HM conditions directly hydroxylated testosterone to 6p-hydroxy testosterone (FIG. 24), which means that the organoids in the HM conditions exhibits functionally mature CYP3A4-mediated drug metabolism activity.

### Experimental Example 5. Prediction of Drug Toxicity Results Using Liver Organoids

The 2D differentiated mature hepatocytes (2D MH) and organoids (HM) in the HM conditions were seeded in a 24-well plate. Each of the drugs (Troglitazone, APAP acetaminophen, Rotenone, and dexamethasone) was continuously diluted from 100-fold Cmax with dimethyl sulfoxide (Sigma; D2650). Three days after seeding, the drug was added daily for 6 days, and toxicity was evaluated by calculating the number of cells using Countess II FL (Life Technology). Then, the organoids were washed with PBS, and fluorescence images were taken with a confocal microscope. Relative intensity was measured using ZEN program (Zeiss) in the same region.

The 2D MH and the organoids in the HM conditions were treated with CYP3A4 and CYP1A2/2E1-mediated hepatotoxic drug [Troglitazone (TRC; T892500) and APAP acetaminophen (Sigma; A5000)] and the 2D MH and organoids were treated with Rotenone (Santa Cruz; sc-203242) exhibiting cytotoxicity and safe dexamethasone as control compounds. As a result of treatment, the toxic response against troglitazone and APAP was different in 2D and 3D models (FIG. 25).

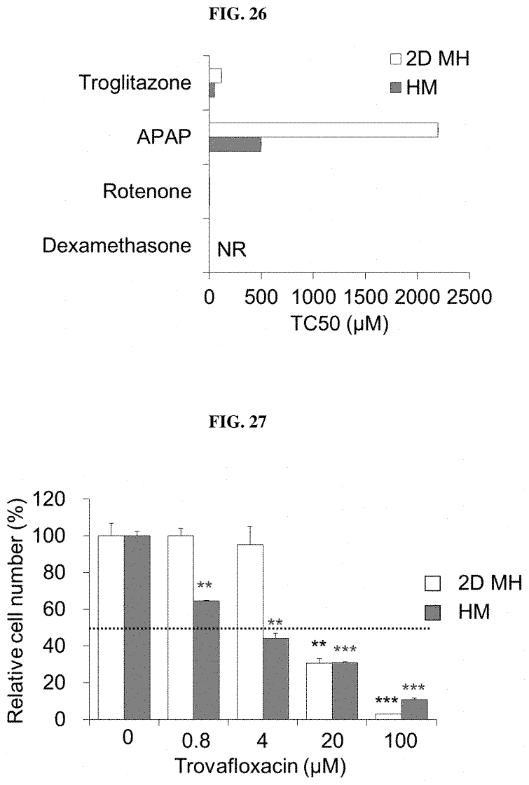

As a result of measuring the toxicity concentration (TC50) based on the cell viability, it was confirmed that the toxicity sensitivity in the organoids was significantly increased compared with the 2D MH (FIG. 26).

Next, the effects of trovafloxacin (Sigma; PZ0015) and levofloxacin (Sigma; 28,266), which are structurally related antibiotics, on the 2D MH and the organoids in the HM conditions were compared. Trovafloxacin has been reported to have a side effect of patient death due to liver failure, and levofloxacin is a non-toxic analog of trovafloxacin.

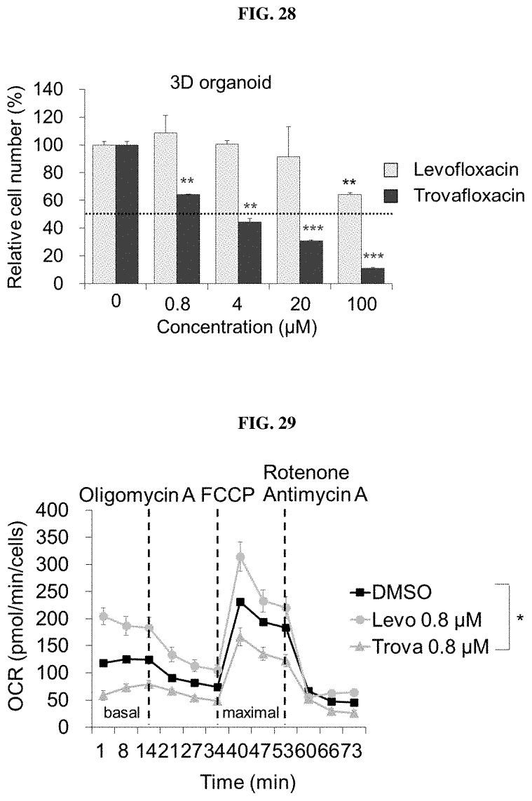

As a result, in the organoids in the HM conditions treated with 0.8 μM and 4 μM (equal to or less than Cmax concentration (4.1 μM)), the number of cells was significantly decreased and the toxicity was detected, but there was little change in the number of cells in the 2D MH (FIG. 27). Not all the drugs were sensitively toxic to the liver organoids: levofloxacin, a non-toxic analog, was non-toxic until Cmax concentration (23.8 μM), and exhibited toxicity only at the highest concentration (100 μM) treated, indicating 36% reduction in the cell viability in the organoids (FIG. 28).

Oxygen consumption rate (OCR) was measured for real-time monitoring of mitochondrial toxicity. Two days before the measurement, the organoids were seeded on XFe 96-well plate (Agilent; 102416-100). The probe cartridge was adjusted overnight in a non-C02 incubator at 37° C. On the assay day, the culture medium was removed and washed with warm assay medium (Agilent Seahorse XF base medium (102353-100) supplemented with 1 mM glutamine, 1 mM pyruvate and 17.5 mM glucose for OCR measurement), and the assay medium was added. The culture dish was placed in a non-CO₂ incubator at 37° C. for 1 hour, and OCR was measured using Seahorse XFe96 Flux Analyzer according to the manufacturer's instructions.

For OCR measurement, ATP synthesis inhibitors (1.5 μM oligomycin and ETC complex V inhibitor), uncoupler (1 μM FCCP), and complex I inhibitor (0.5 μM Rotenone) plus complex III inhibitor (0.5 μM antimycin A) were sequentially added at designated time points. The measured value was normalized to the number of surviving cells measured by Cell Counting Kit-8 (Dogindo; CK04-01).

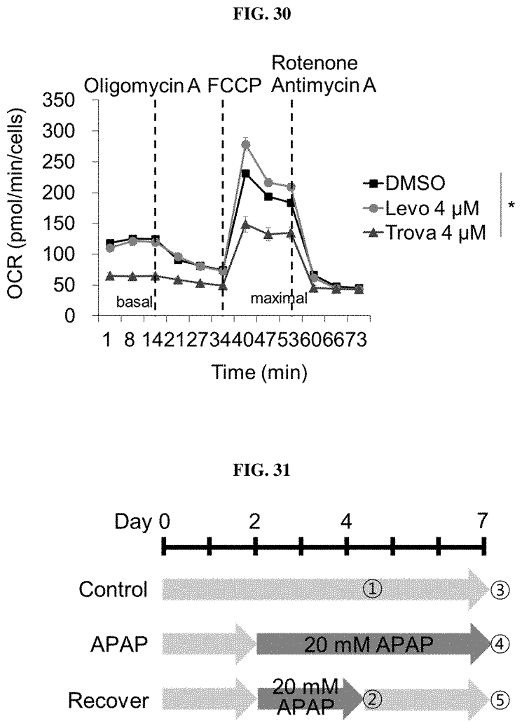

As a result, in the case of treatment with small amounts of trovafloxacin, such as 0.8 μM (FIG. 29) and 4 μM (FIG. 30), damage to mitochondrial respiration was clearly demonstrated through reduction of OCR in the organoids.

Accordingly, it was confirmed that the liver organoids in the HM conditions exhibited sensitivity and evaluation accuracy to drug toxicity, which was inherent in human liver tissues, and thus can be used as a liver model for evaluating drug toxicity.

### Experimental Example 6. Analysis of Regeneration and Inflammatory Response of Liver Organoids

The organoids in the HM conditions were treated with 20 mM APAP for 60 hours on day 2 after seeding, and the medium was replaced with a new HM medium for recovery or continuously treated with 20 mM APAP until day 7. Time-lapsed images were taken at 37° C. under 5% CO₂ at 30-minute intervals with an Olympus IX83 inverted microscope. The diameter of the organoid was measured using ImageJ program at the designated time point from the time lapsed image. Fluorescence images were taken with a confocal microscope. In addition, the organoids were cultured for 30 minutes with 7.5 μM dihydroethidium (Sigma; D7008) for detecting ROS, 30 μM monochlorobimane (Sigma; 69899) for measuring GSH content, and 2.5 μM Syto11 (Thermo Fisher; S7573) for staining nuclei.

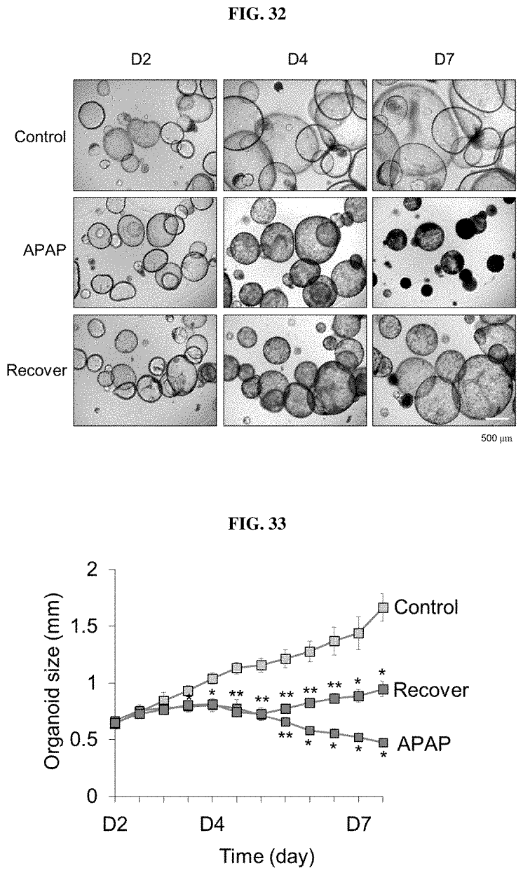

After treatment with high-dose APAP, the recoverability and the inflammatory response of the organoids in the HM conditions were analyzed (FIG. 31). After 7 days of daily treatment with 20 mM of APAP, the organoids showed severe morphological damage, but it was confirmed that the organoids, which were treated with the APAP on day 2 for 60 hours and in which a new HM medium was then replaced on day 4.5, recovered on day 7 (FIG. 32). As a result of measuring the size of the organoids, it was also confirmed that the organoids, which were treated with the APAP on day 2 for 60 hours and in which a new HM medium was then replaced on day 4.5, recovered on day 7 (FIG. 33).

In addition, the expressions of high-mobility group protein 1 (HMGB1), which is a protein involved in the detection of ROS and the inflammatory response of cells, ki67, which is a marker indicating cell proliferation, E-cadherin, which is an epithelial marker, LC3B, which is an autophagy marker, and Tom20, a mitochondrial marker, were analyzed at protein level according to the method described in Experimental Example 1. The used antibodies are as listed in Table 8 below.

TABLE 8

| Antibodies | Catalog No. | Company | Dilution |
|---|---|---|---|
| anti-HMGB1 | Ab79823 | Abcam | 1:200 |
| anti-Ki67 | Ab15580 | Abcam | 1:100 |
| anti-E-cadherin | 610181 | BD biosciences | 1:200 |
| anti-LC3B | #2775 | Cell signaling technology | 1:200 |
| anti-Tom20 | sc-17764 | Santacruz | 1:100 |

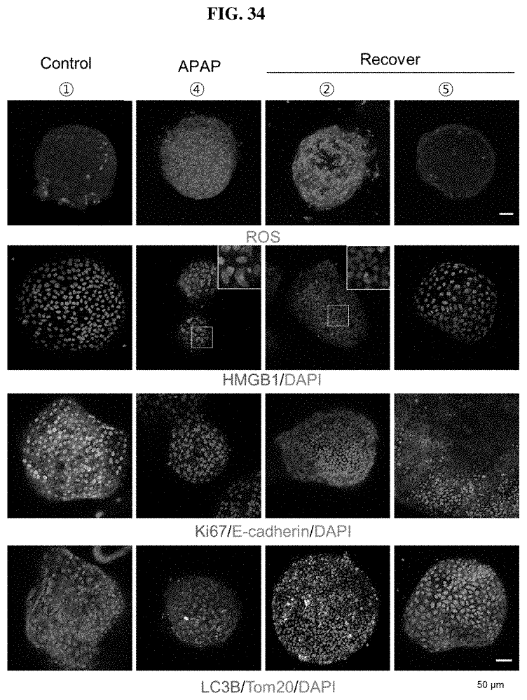

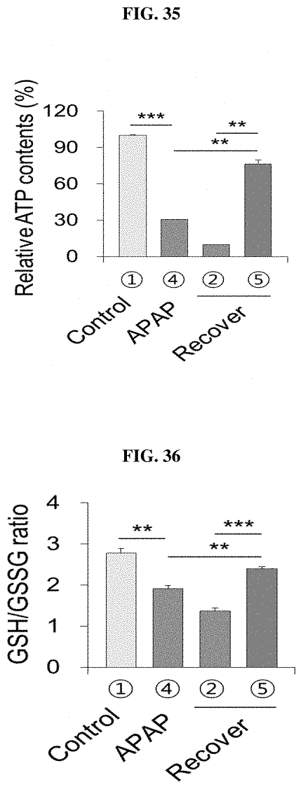

As a result, increased reactive oxygen species (ROS) by the treatment with APAP was detected, and migration to the cytoplasm appeared while the expression of HMGB1 was reduced, and the expression amounts of Ki67, E-cadherin, and Tom20 were also reduced. However, it was confirmed that the expression amounts were recovered to a level similar to that of the control group after being replaced with a new HM medium (FIG. 34). Meanwhile, the expression of LC3B was more strongly induced in the case of treatment with APAP for 60 hours than the case of treatment with APAP for 5 days (FIG. 34), which shows a pattern similar to the cases of ATP content (FIG. 35) and GSH (glutathione)/GSSG (Glutathione disulfide) ratio (FIG. 36).

In order to compare the expression amounts of inflammatory response-related proteins in the organoids treated with APAP daily for 7 days and the organoids which were treated with the APAP on day 2 for 60 hours and in which a new HM medium was then replaced on day 4.5, qRT-PCR was performed according to the method described in Experimental Example 2.1, and the expression amounts were represented in comparison with a base level. The used primer sequences are as listed in Table 9 below.

TABLE 9

| | | SEQ |
|---|---|---|
| Primer | Base sequence | ID NO. |
| IL-1β_F | AATCTGTACCTGTCCTGCGTGTT | 35 |
| IL-1β_R | TGGGTAATTTTTGGGATCTACACT | 36 |
| IL-6_F | GGTACATCCTCGACGGCATCT | 37 |
| IL-6_R | GTGCCTCTTTGCTGCTTTCAC | 38 |
| IL-8_F | CTTGGCAGCCTTCCTGATTT | 39 |
| IL-8_R | TTCTTTAGCACTCCTTGGCAAAA | 40 |
| IL-10_F | GCCTAACATGCTTCGAGATC | 41 |
| IL-10_R | TGATGTCTGGGTCTTGGTTC | 42 |
| TNFa_F | GGAGAAGGGTGACCGACTCA | 43 |
| TNFa_R | CTGCCCAGACTCGGCAA | 44 |
| FasL_F | TCTGGAATGGGAAGACACC | 45 |
| FasL_R | CACATCTGCCCAGTAGTGC | 46 |

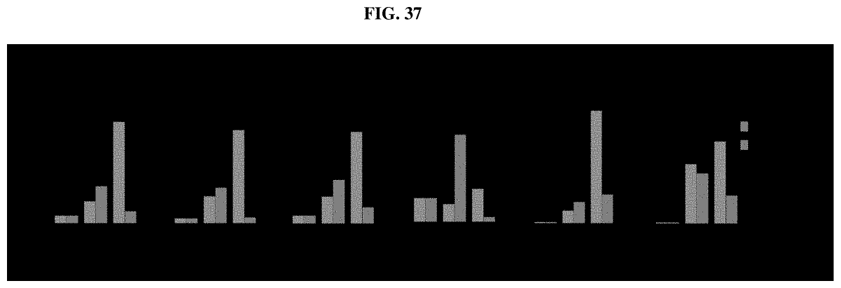

As a result, the expression of IL-10, an anti-inflammatory mediator, was significantly increased in the organoids which were treated with the APAP for 60 hours and in which a new HM medium was then replaced on day 4.5, but the expressions of proinflammatory mediators (IL-1p, IL-6, and IL-8), and pathological mediators (TNF-α and FasL) were strongly induced in the organoids which were treated with the APAP for 7 days (FIG. 37).

Accordingly, it was confirmed that the liver organoids in the HM conditions can be used as a liver model for understanding regeneration and inflammatory responses after hepatotoxicity damage.

### Experimental Example 7. Fatty Liver Modeling Using Liver Organoids

The organoids in the HM conditions were seeded on a 24-well plate in order to be induced into liver organoids with steatosis. Three days after seeding, 0.5 mM oleate (Sigma; 07501) combined with 12% fatty acid-free BSA (Sigma; A8806) and 0.25 mM palmitate (Sigma; P9767) were added for 3 days, and images of accumulated lipid droplets were observed under a microscope. For Nile red staining, the samples were washed with PBS and fixed with 4% PFA at 4° C. overnight. The organoids were washed with PBS and treated with 10 μg/ml Nile red solution (Thermo Fisher; N1142) at room temperature for 5 minutes. Fluorescence images were taken with a confocal microscope.

For the analysis of triglyceride concentration, the steatosis-induced organoids were analyzed by using triglyceride assay kit (Abcam; ab65336) according to the manufacturer's instructions. The organoids were homogenized for 5 minutes using 1 mL of a 5% NP-40 solution under the condition of heated to 80 to 100° C. The pellets were diluted 10-fold with dilution water before starting the analysis. Absorbance was measured at 570 nm by using SpectraMax microplate reader.

For drug screening, the organoids were treated with 151 chemical substances at a concentration of 10 μM in the autophagy library (Selleckchem; At L2600) during the hepatic steatosis induction period. Then, the organoids were stained with Nile red and fluorescence images were analyzed with a confocal microscope.

Meanwhile, in order to confirm the effect on the induction of hepatic steatosis, the organoids in the HM conditions were treated with BSA, FA, FA+etomoxir, FA+L-carnitine, and FA+metformin, respectively, and the results were compared.

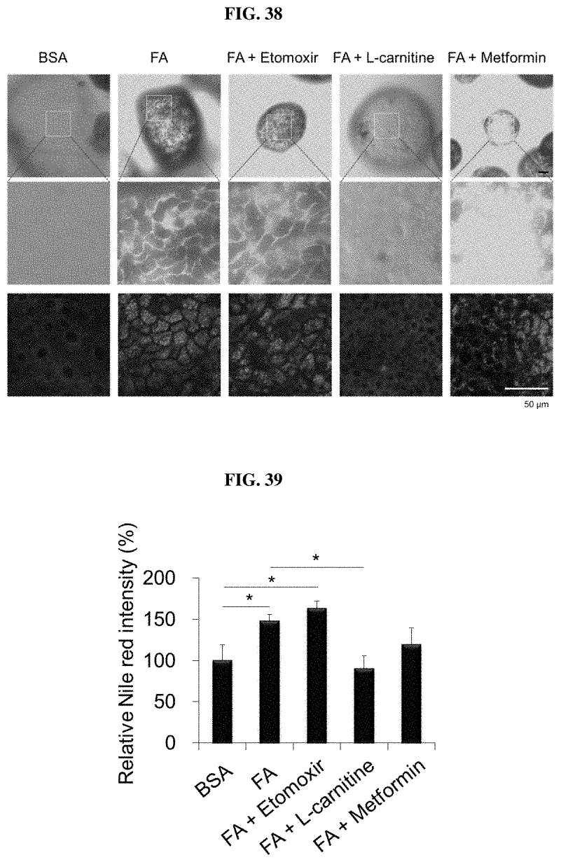

When the organoids were additionally treated with etomoxir, which is an irreversible inhibitor of carnitine palmitoyltransferase-1 (CPT1) which blocks a carnitine shuttle that transports fatty acids (FA) to a mitochondrial membrane for β-oxidation, it was confirmed that the induction of steatosis was promoted. That is, FA plus etomoxir treatment showed significant accumulation of intracellular lipid droplets observed by a bright field microscope and Nile red staining (FIGS. 38 and 39).

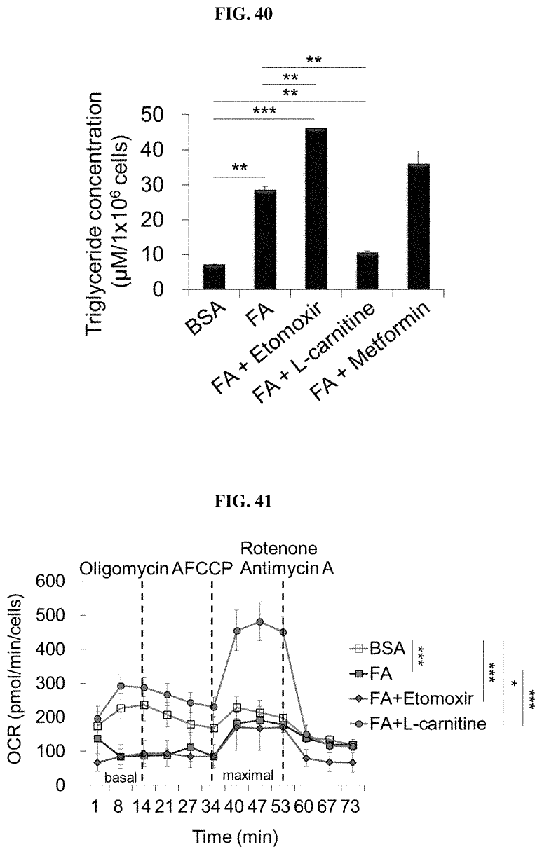

The intracellular triglyceride concentration was significantly increased by FA plus etomoxir treatment compared with the BSA control or FA alone treatment group (FIG. 40). Functionally, mitochondrial respiration measured by OCR was significantly reduced by FA plus etomoxir treatment (FIG. 41). On the contrary, it was confirmed that for the FA plus L-carnitine treatment group, the lipid accumulation was significantly reduced and the mitochondrial respiration was recovered by promoting the carnitine shuttle of mitochondria as compared with the FA alone treatment group. In addition, as a result of treating FA with metformin, an antidiabetic drug that reduces hepatic steatosis, lipid accumulation was slightly reduced, but the concentration of triglycerides was not reduced compared with the case of using FA alone.

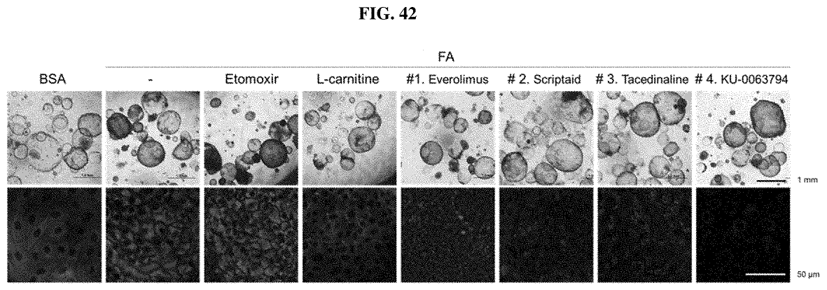

The phenotype of hepatic steatosis and the top four compounds capable of restoring lipid accumulation (Everolimus, Scriptaid, Tacedinalin, and KU-0063794) were identified through drug screening (FIG. 42).

qRT-PCR was performed according to the method described in Experimental Example 2.1 in order to compare the expression levels of CD36 which is a liver fatty acid translocase, SREBP which is a fatty acid production related factor, and CPT1 which is associated with β-oxidation when the hepatic steatosis organoids were treated with the four compounds. The used primer sequences are as listed in Table 10 below.

TABLE 10

| Primer | Base sequence | SEQ ID NO. |
|---|---|---|
| CD36_F | AGATGCAGCCTCATTTCCAC | 47 |
| CD36_R | GCCTTGGATGGAAGAACAAA | 48 |
| SREBP_F | TCAGCGAGGCGGCTTTGGAGCAG | 49 |
| SREBP_R | CATGTCTTCGATGTCGGTCAG | 50 |
| CPTl_F | CCTACCACGGGTGGATGTTC | 51 |
| CPTl_R | CAACATGGGTTTTCGGCCTG | 52 |

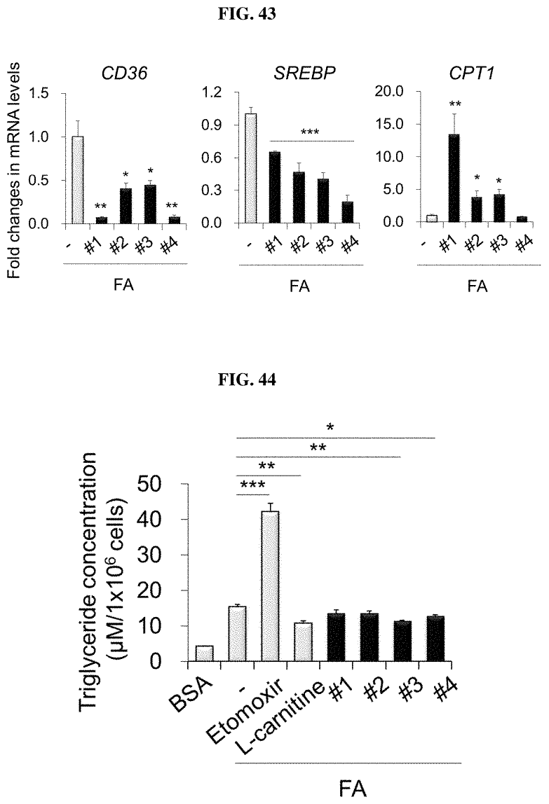

The expression amounts of CD36 and SREBP were both reduced when treated with each of four kinds of compounds, but the expression amount of CPT1 was increased when treated with each of four kinds of compounds (FIG. 43). In addition, the concentration of triglycerides was also reduced when treated with the four kinds of compounds (FIG. 44).

Accordingly, it was confirmed that the liver organoids in the HM conditions can be induced as a fatty liver model, which can be used as a liver model for screening therapeutic agents for fatty liver.

III. Direct Differentiation of Liver Organoids from Hepatic Endoderm Cells

### Example 4. Preparation of Liver Organoids According to Medium

The hepatic endoderm (HE) cells in Example 2 differentiated from CRL-2097-derived human iPSCs via definitive endoderm (DE) cells were isolated into single cells, and the single cells were 3D-cultured in each of MH medium (condition b), HM medium (condition c), EM medium (condition d), and DM medium (condition e) to produce liver organoids (see New protocol II in FIG. 1).

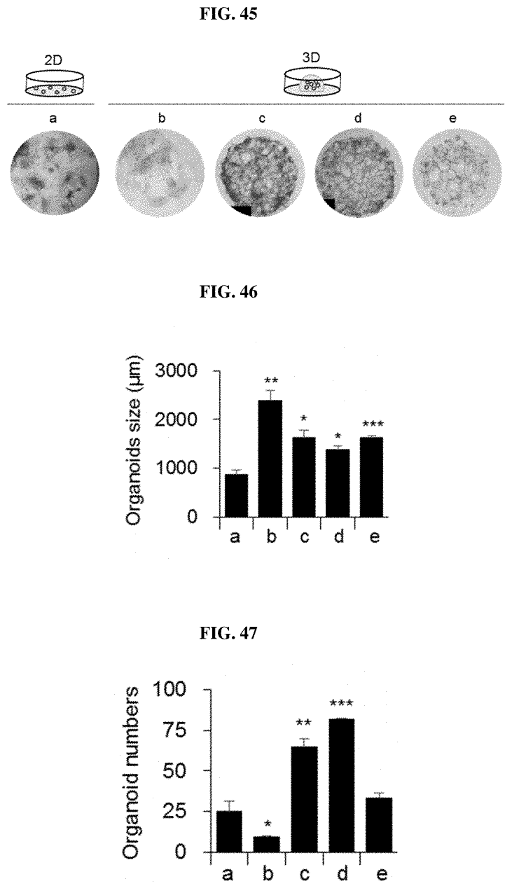

Twenty five days after starting the 3D culture, images of the organoids cultured in each medium were taken (FIG. 45), and the sizes (FIG. 46) and number (FIG. 47) of the generated organoids were quantified and compared with 2D mature hepatocytes (MH) produced according to the conventional protocol of Example 3.1 (condition a).

As a result, compared with the conventional 2D method (condition a), it was confirmed that the sizes of the organoids cultured in the 3D culture medium were all increased, and the number of the organoids cultured in the 3D culture medium was increased by 2.5 times in the HM medium and 3.3 times in the EM medium.

### Experimental Example 8. Evaluation of Proliferation Ability of Liver Organoids

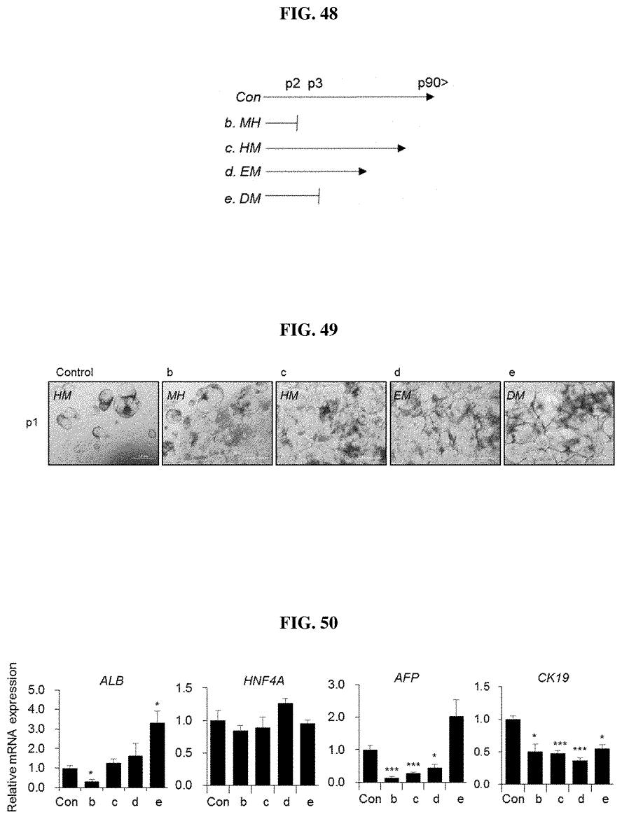

The liver organoids obtained in Example 3.2 as a control group were subcultured in the HM medium, and the liver organoids produced in each of the MH medium (condition b), HM medium (condition c), EM medium (condition d), and DM medium (condition e) were subcultured. As a result, it was confirmed that the liver organoids produced in the MH medium were not able to be proliferated at least twice subcultures (p2), and the liver organoids produced in the DM medium were not able to be proliferated at least three times subcultures (p3) (FIG. 48).

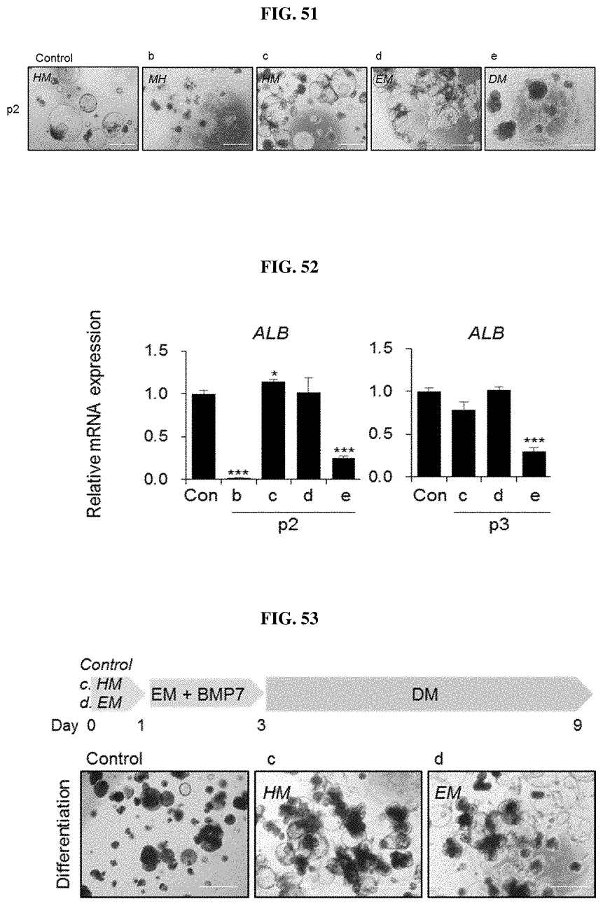

The liver organoids produced in each of the control group, the MH medium, the HM medium, the EM medium, and the DM medium were subcultured once (p1) and twice, respectively, and then images were captured (FIGS. 49 and 51). In addition, in order to compare the expression amounts of hepatocyte-specific markers (ALB and HNF4A) with the expression amounts of fetal liver/precursor-specific markers (AFP and CK19), in p1, qRT-PCR was performed according to the method described in Experimental Example 2.1. The used primer sequences are as listed in Table 11 below.

| | | SEQ |
|---|---|---|
| Primer | Base sequence | ID NO. |
| ALB_F | TTTATGCCCCGGAACTCCTTT | 17 |
| ALB_R | AGTCTCTGTTTGGCAGACGAA | 18 |
| AFP_F | AGCTTGGTGGTGGATGAAAC | 53 |
| AFP_R | CCCTCTTCAGCAAAGCAGAC | 54 |
| CK19_F | CGCGGCGTATCCGTGTCCTC | 15 |
| CK19_R | AGCCTGTTCCGTCTCAAACTTGGT | 16 |
| HNF4A_F | GGCCAAGTACATCCCAGCTTT | 55 |
| HNF4A_R | CAGCACCAGCTCGTCAAGG | 56 |

As a result, it was confirmed that the expression of ALB and HNF4A in the liver organoids produced in the HM medium was similar to that of the control group, and the expression amounts of AFP and CK19 were decreased by 3 times and 2 times, respectively, as compared with those of the control group (FIG. 50). This means that when the liver organoids are produced from the hepatic endoderm using the HM medium, the characteristics of the immature hepatocyte exhibited by the liver organoids of the control group are reduced.

Meanwhile, it was confirmed that in the case of the liver organoids produced in the DM medium, the expression amount of ALB was the highest in p1 compared with other conditions, but the expression amount of ALB was significantly reduced compared with the control group as subculturing progressed, and in the case of the liver organoids produced in the HM medium and the EM medium, the expression amount of ALB was maintained similarly to that of the control group (FIG. 52).

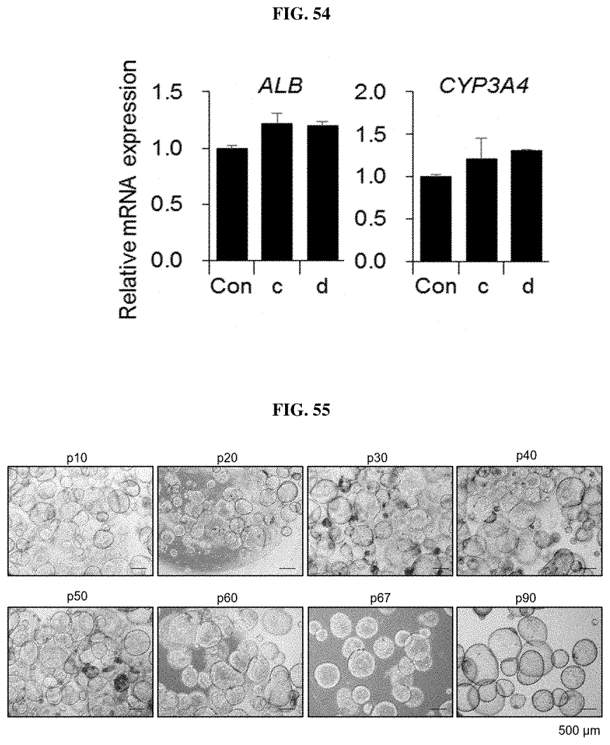

In addition, when the liver organoids produced in the HM medium and EM medium were cultured in the EM medium containing 25 ng/mL of BMP7 for two days, and then cultured in the DM medium for 6 days to be further differentiated (FIG. 53), ALB and CYP3A4 were expressed at levels similar to those of the control group, thereby confirming their functionality as mature hepatocytes (FIG. 54).

### Experimental Example 9. Characterization of Liver Organoids

It was confirmed that the liver organoids produced in the HM medium were still alive even after 90 subcultures (p90) (FIG. 55).

In addition, changes after the freezing and thawing processes of the liver organoids produced in the HM medium were confirmed. Specifically, for cryopreservation, the subcultured liver organoids were mixed with mFreSR (Stem Cell Technology; 05855) and freezing/thawing were carried out according to standard procedures. After thawing, 10 μM Y-27632 (Tocris; 1254) was added to the medium for 3 days. Then, the number of surviving cells was counted.

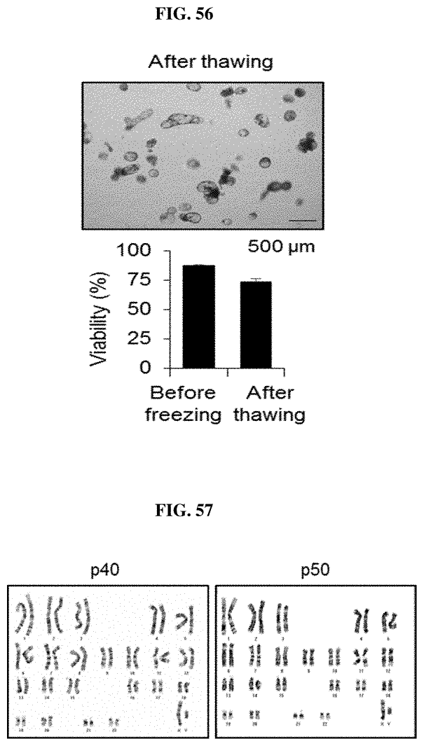

As a result, it was confirmed that even after the freezing and thawing processes, the shape of the liver organoid was maintained, and the cell viability was maintained at 73±2.56% (FIG. 56).

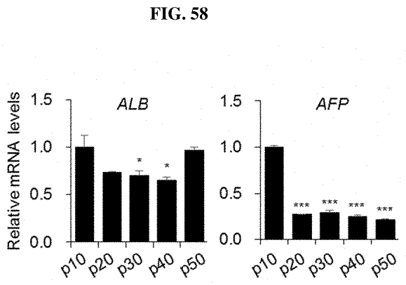

Meanwhile, it was confirmed that the normal karyotype was also maintained in p50 (FIG. 57), and it was confirmed that the expression of ALB, a hepatocyte-specific marker, was maintained up to p50, and the expression of AFP, a fetal/precursor marker, was reduced (FIG. 58). This means that the functionality of mature hepatocytes was maintained even after multiple subcultures.

### Experimental Example 10. Characterization of HM Medium

It was confirmed that the liver organoids produced in the HM medium according to the present invention had proliferation and differentiation ability similar to those of the liver organoids produced in the EM medium without expensive R-spondin, and as shown in Table 1, experiments were performed to confirm the effects of bFGF, OSM, and ITS, which are components distinguished from known liver organoid culture media (MH medium, EM medium, and DM medium).

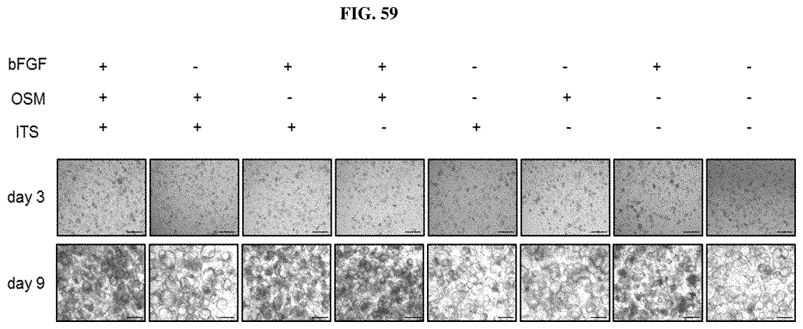

In order to find the main components affecting the expandable characteristics of the liver organoids, each of bFGF, OSM, and ITS, or a combination thereof was removed during the generation of organoids (FIG. 59) or the later subculture process (FIG. 62), and the results were verified.

In the process of generating organoids, when each of bFGF, OSM, and ITS was removed alone, it did not significantly affect in the initial stage, and when the two components at a time were removed, the organoid generation efficiency decreased (FIG. 60, day 3), that is, it was confirmed that in the initial stage of organoid generation, three components were functionally complementary to each other.

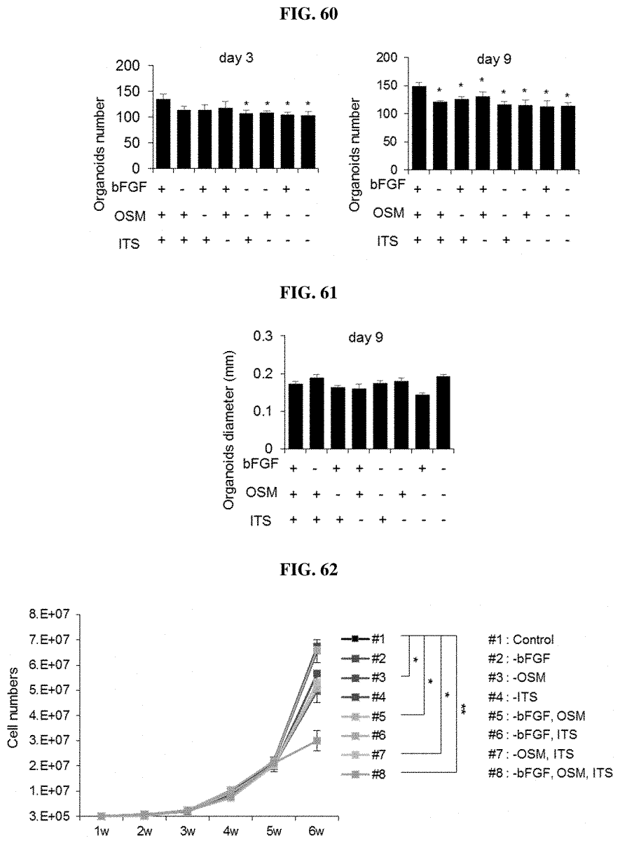

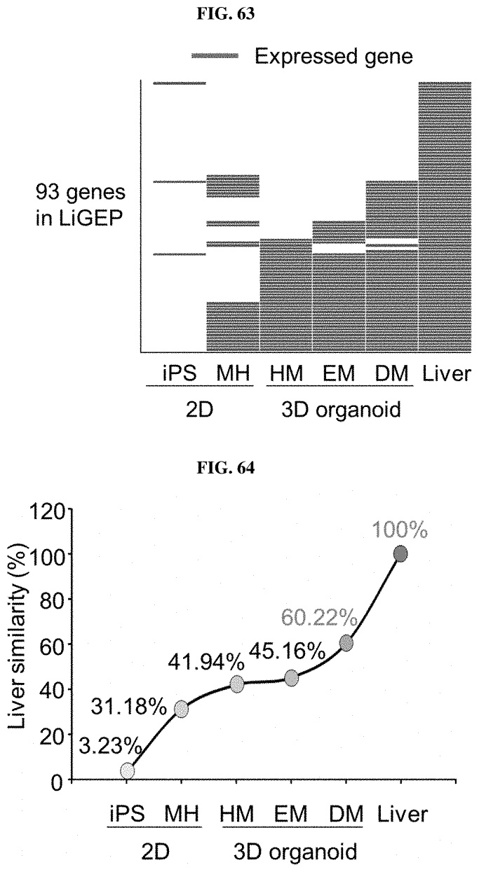

Meanwhile, in the process of generating organoids, the number of organoids generated under all the conditions was reduced on day 9 compared with the control group (FIG. 60, day 9). That is, it was confirmed that the generation of organoids was inhibited when even one of the three components was absent in the process of generating organoids. However, the size of the generated organoids was not affected (FIG. 61).

As a result of removing each component in the later subculture process (p40-p45) of the organoids for 6 weeks, it was confirmed that a significant cell proliferation inhibition appeared in the conditions of #3 (-OSM), #5 (-bFGF, OSM), #7 (-OSM, ITS), and #8 (-bFGF, OSM, ITS) in comparison with the control group (FIG. 62). In particular, when all three components were removed, the most significant proliferation inhibition effect was shown compared with the control group, and OSM among the three components is expected to be the most important component.

V. Analysis of Genes Expressed in Liver Organoids

### Example 5. RNA-sequencing and LiGEP Analysis

RNA sequencing was performed by using Illumina HiSeq 2500 instrument, the quality of raw reads (both ends of 100 bp) was verified, and the low-quality base and adapter sequences were filtered. Quality tests were performed by using FastQC. Low-quality reads and bases were filtered in the data set before read mapping. Cutadapt (v1.13) and Sickle (v1.33) were used to eliminate adapter contamination and low-quality reads. The treated reads were aligned to human reference genome (hg19) by using HISAT2 (v2.1.0). Gene expression was quantified by using the values of FPKM. Expression values of 93 genes of the liver-specific gene expression panel (LiGEP) known in Kim D S, et al. A liver-specific gene expression panel predicts the differentiation status of in vitro hepatocyte models. Hepatology 2017; 66:1662-1674 were measured, and the similarity to human liver tissue was calculated by using LiGEP algorithm disclosed in Korean Patent No. 10-1920795.

### Experimental Example 11. Analysis of Biomarkers Expressed in Liver Organoids

Among 93 adult liver tissue-specifically expressed genes of the liver specific gene expression panel (LiGEP) developed by the present inventors to evaluate the differentiation state of the liver model based on RNA sequencing and to indicate the liver similarity, biomarkers of genes expressed in the 2D hepatocytes (MH) obtained in Example 3.1, the liver organoids (HM) obtained in Example 3.2, the liver organoids (EM) in which the liver organoids obtained in Example 3.2 were cultured in the EM, or the liver organoids produced by culturing the liver organoids obtained in Example 3.2 in the EM and then culturing in the DM were identified, and the similarity to the liver tissue was measured (FIG. 64). As a result, the 2D MH exhibited the similarity of 31.18%, the JIM exhibited the similarity of 41.94%, the EM exhibited the similarity of 45.16%, and the DM exhibited the similarity of 60.22%. The results of comparing 93 liver tissue-specific genes with genes expressed in each liver organoid are as listed in Table 12 below.

TABLE 12

| | Symbol | iPS | 2D MH | HM | EM | DM | Liver |
|---|---|---|---|---|---|---|---|
| 1 | KLKB1 | ✓ | | | | | ✓ |
| 2 | SLC25A47 | | | | | | ✓ |
| 3 | SLC13A5 | | | | | | ✓ |
| 4 | SDS | | | | | | ✓ |
| 5 | RDH16 | | | | | | ✓ |
| 6 | PON3 | | | | | | ✓ |
| 7 | PON1 | | | | | | ✓ |
| 8 | MASP2 | | | | | | ✓ |
| 9 | ITIH4 | | | | | | ✓ |
| 10 | ITIH3 | | | | | | ✓ |
| 11 | HSD17B13 | | | | | | ✓ |
| 12 | HSD11B1 | | | | | | ✓ |
| 13 | HRG | | | | | | ✓ |
| 14 | HPR | | | | | | ✓ |
| 15 | HGFAC | | | | | | ✓ |
| 16 | HAO1 | | | | | | ✓ |
| 17 | GNMT | | | | | | ✓ |
| 18 | FMO3 | | | | | | ✓ |
| 19 | F9 | | | | | | ✓ |
| 20 | CYP4F2 | | | | | | ✓ |
| 21 | CYP2E1 | | | | | | ✓ |
| 22 | CYP2D6 | | | | | | ✓ |
| 23 | CYP1A2 | | | | | | ✓ |
| 24 | CP | | | | | | ✓ |
| 25 | CFHR1 | | | | | | ✓ |
| 26 | C9 | | | | | | ✓ |
| 27 | C6 | | | | | | ✓ |
| 28 | C5orf27 | | | | | | ✓ |
| 29 | AQP9 | | | | | | ✓ |
| 30 | APOF | | | | | | ✓ |
| 31 | ADH6 | | | | | | ✓ |
| 32 | APOC4 | | | | | | ✓ |
| 33 | SERPIND1 | | ✓ | | | | ✓ |
| 34 | SULT2A1 | | ✓ | | | | ✓ |
| 35 | MAT1A | ✓ | ✓ | | | ✓ | ✓ |
| 36 | TDO2 | | ✓ | | | ✓ | ✓ |
| 37 | SERPINC1 | | ✓ | | | ✓ | ✓ |
| 38 | ITIH1 | | ✓ | | | ✓ | ✓ |
| 39 | AHSG | | ✓ | | | ✓ | ✓ |
| 40 | C8B | | ✓ | | | ✓ | ✓ |
| 41 | SAA4 | | | | | ✓ | ✓ |
| 42 | LEAP2 | | | | | ✓ | ✓ |
| 43 | HAMP | | | | | ✓ | ✓ |
| 44 | FMO5 | | | | | ✓ | ✓ |
| 45 | CYP2A6 | | | | | ✓ | ✓ |
| 46 | CFHR2 | | | | | ✓ | ✓ |
| 47 | AGXT | | | | | ✓ | ✓ |
| 48 | C8G | | | | | ✓ | ✓ |
| 49 | ALB | | ✓ | | ✓ | ✓ | ✓ |
| 50 | APOH | | ✓ | | ✓ | ✓ | ✓ |
| 51 | SLC22A1 | | | | ✓ | ✓ | ✓ |
| 52 | LECT2 | | | | ✓ | ✓ | ✓ |
| 53 | ADH1A | | | | ✓ | ✓ | ✓ |
| 54 | ADH4 | | | | ✓ | ✓ | ✓ |
| 55 | SLC38A4 | | | ✓ | ✓ | | ✓ |
| 56 | AFM | | ✓ | ✓ | ✓ | | ✓ |
| 57 | SLC10A1 | | ✓ | ✓ | | ✓ | ✓ |
| 58 | CYP8B1 | | | ✓ | | | ✓ |
| 59 | CXCL2 | | | ✓ | | ✓ | ✓ |
| 60 | SLCO1B1 | ✓ | | ✓ | ✓ | ✓ | ✓ |
| 61 | UGT2B15 | | | ✓ | ✓ | ✓ | ✓ |
| 62 | UGT2B10 | | | ✓ | ✓ | ✓ | ✓ |
| 63 | TAT | | | ✓ | ✓ | ✓ | ✓ |
| 64 | SLC2A2 | | | ✓ | ✓ | ✓ | ✓ |
| 65 | SERPINA6 | | | ✓ | ✓ | ✓ | ✓ |
| 66 | SERPINA4 | | | ✓ | ✓ | ✓ | ✓ |
| 67 | HABP2 | | | ✓ | ✓ | ✓ | ✓ |
| 68 | GC | | | ✓ | ✓ | ✓ | ✓ |
| 69 | F12 | | | ✓ | ✓ | ✓ | ✓ |
| 70 | CYP2C9 | | | ✓ | ✓ | ✓ | ✓ |
| 71 | CYP2C8 | | | ✓ | ✓ | ✓ | ✓ |
| 72 | CPB2 | | | ✓ | ✓ | ✓ | ✓ |
| 73 | CFHR3 | | | ✓ | ✓ | ✓ | ✓ |
| 74 | BAAT | | | ✓ | ✓ | ✓ | ✓ |
| 75 | AKR1C4 | | | ✓ | ✓ | ✓ | ✓ |
| 76 | APCS | | | ✓ | ✓ | ✓ | ✓ |
| 77 | VTN | | ✓ | ✓ | ✓ | ✓ | ✓ |
| 78 | UGT2B4 | | ✓ | ✓ | ✓ | ✓ | ✓ |
| 79 | SERPINA11 | | ✓ | ✓ | ✓ | ✓ | ✓ |
| 80 | PROC | | ✓ | ✓ | ✓ | ✓ | ✓ |
| 81 | ITIH2 | | ✓ | ✓ | ✓ | ✓ | ✓ |
| 82 | HPX | | ✓ | ✓ | ✓ | ✓ | ✓ |
| 83 | FGL1 | | ✓ | ✓ | ✓ | ✓ | ✓ |
| 84 | FGG | | ✓ | ✓ | ✓ | ✓ | ✓ |
| 85 | FGB | | ✓ | ✓ | ✓ | ✓ | ✓ |
| 86 | FGA | | ✓ | ✓ | ✓ | ✓ | ✓ |
| 87 | F2 | | ✓ | ✓ | ✓ | ✓ | ✓ |
| 88 | C8A | | ✓ | ✓ | ✓ | ✓ | ✓ |
| 89 | C4BPB | | ✓ | ✓ | ✓ | ✓ | ✓ |
| 90 | APOB | | ✓ | ✓ | ✓ | ✓ | ✓ |
| 91 | APOA2 | | ✓ | ✓ | ✓ | ✓ | ✓ |
| 92 | AMBP | | ✓ | ✓ | ✓ | ✓ | ✓ |
| 93 | ANGPTL3 | | ✓ | ✓ | ✓ | ✓ | ✓ |

Meanwhile, 16 genes that are always expressed in five different sample groups of liver organoids produced in the JIM medium obtained in Example 4 were identified (Table 13).

TABLE 13

| | Symbol | HM | Liver |
|---|---|---|---|
| 1 | AMBP | √ | √ |
| 2 | APOA2 | √ | √ |
| 3 | APOB | √ | √ |
| 4 | CYP8B1 | √ | √ |
| 5 | F2 | √ | √ |
| 6 | FGA | √ | √ |
| 7 | FGB | √ | √ |
| 8 | FGG | √ | √ |
| 9 | HABP2 | √ | √ |
| 10 | ITIH2 | √ | √ |
| 11 | PROC | √ | √ |
| 12 | SERPINA11 | √ | √ |
| 13 | SERPINA4 | √ | √ |
| 14 | SLC2A2 | √ | √ |
| 15 | UGT2B15 | √ | √ |
| 16 | VTN | √ | √ |

In addition, differences between genes expressed in each of the liver organoids obtained in the 2D MH medium, EM medium, and DM medium of Example 4 and genes expressed in the liver organoids (JIM) produced in the JIM medium are shown in Tables 14 to 16 below.

TABLE 14

| | Symbol | 2DMH | HM | Liver |
|---|---|---|---|---|
| 1 | SLC2A2 | | √ | √ |
| 2 | CYP2C9 | | √ | √ |
| 3 | CYP2C8 | | √ | √ |
| 4 | CYP8B1 | | √ | √ |
| 5 | UGT2B15 | | √ | √ |
| 6 | UGT2B10 | | √ | √ |
| 7 | AKR1C4 | | √ | √ |
| 8 | SLC38A4 | | √ | √ |
| 9 | CXCL2 | | √ | √ |
| 10 | TAT | | √ | √ |
| 11 | SLCO1B1 | | √ | √ |
| 12 | BAAT | | √ | √ |
| 13 | SERPINA4 | | √ | √ |
| 14 | HABP2 | | √ | √ |
| 15 | F12 | | √ | √ |
| 16 | CPB2 | | √ | √ |
| 17 | SERPINA6 | | √ | √ |
| 18 | GC | | √ | √ |
| 19 | CFHR3 | | √ | √ |
| 20 | APCS | | √ | √ |

TABLE 15

| | Symbol | HM | EM | Liver |
|---|---|---|---|---|
| 1 | SLC10A1 | √ | | √ |
| 2 | CYP8B1 | √ | | √ |
| 3 | CXCL2 | √ | | √ |

TABLE 16

| | Symbol | HM | DM | Liver |
|---|---|---|---|---|
| 1 | SLC38A4 | √ | | √ |
| 2 | AFM | √ | | √ |
| 3 | CYP8B1 | √ | | √ |

1. A medium composition for differentiation of liver organoids, comprising a basic fibroblast growth factor (bFGF), oncostatin M (OSM), and insulin-transferrin-selenium (ITS).

2. The medium composition of claim 1, further comprising at least one selected from the group consisting of PS, GlutaMAX, HEPES, N2 supplement, N-acetylcysteine, [Leu15]-Gastrin I, an epidermal growth factor (EGF), a hepatocyte growth factor (HGF), vitamin A-free B27 supplement, A83-01, nicotinamide, forskolin, dexamethasone, and a combination thereof.

3. The medium composition of claim 1, which is applied to hepatic endoderm or hepatocytes differentiated from stem cells.

4. A method for producing liver organoids, comprising culturing hepatic endoderm cells or hepatocytes in the medium composition described in claim 1.

5. The method of claim 4, wherein the hepatic endoderm cells or hepatocytes are differentiated from stem cells.

6. The method of claim 4, wherein the hepatic endoderm cells or the hepatocytes are cultured in three dimensions to differentiate into liver organoids.

7. The method of claim 4, wherein the hepatic endoderm cells are isolated into single cells and then embedded in a matrix to differentiate into liver organoids.

8. The method of claim 4, wherein the liver organoids are able to be subcultured at least 90 times.

9. Expandable liver organoids, expressing AMBP, APOA2, APOB, CYP8B1, F2, FGA, FGB, FGG, HABP2, ITIH2, PROC, SERPINA11, SERPINA4, SLC2A2, UGT2B15, and VTN as liver-specific genetic markers.

10. The expandable liver organoids of claim 9, further expressing one or more liver-specific genetic markers selected from the group consisting of SLC2A2, CYP2C9, CYP2C8, UGT2B10, AKR1C4, SLC38A4, CXCL2, TAT, SLCO1B1, BAAT, F12, CPB2, SERPINA6, GC, CFHR3, APCS, SLC10A, CXCL2, SLC38A4, and AFM.

11. The expandable liver organoids of claim 9, which are able to be subcultured at least 90 times.

12. The expandable liver organoids of claim 9, which are differentiated from stem cells.

13. The expandable liver organoids of claim 9, which are produced by culturing hepatic endoderm or hepatocytes differentiated from stem cells in a medium composition comprising basic fibroblast growth factor (bFGF), oncostatin M (OSM), and insulin-transferrin-selenium (ITS).

14. The expandable liver organoids of claim 9, wherein, when the expression amount of liver tissue-specific gene measured in the liver organoids is applied to Equation 1 below, the similarity to liver tissue is at least 40%:
$${D}_{i} = ( 1 - \frac{{B}_{i} + {C}_{i}}{{A}_{i} + {B}_{i} + {C}_{i}} ) \times 100 ( \% ) \qquad [ Equation 1 ]$$
wherein, in Equation 1 above, Aᵢ, Bᵢ, and Cᵢ are I(yᵢ−Uᵢ>0)·I(zᵢ>0), I(yᵢ−Uᵢ≤0)·I(zᵢ>0), and I(yᵢ−Uᵢ>0)·I(zᵢ≤0), respectively, wherein yᵢ is the value of fragments per kilobase of exon model per million mapped reads (FPKM) of the i-th gene of the liver tissue sample, wherein Uᵢ is the upper limit value of 100×(1−α)% confidence interval of the Wilcoxon signed-rank test, and wherein zᵢ is the difference (uᵢ−Uᵢ) between the FPKM value (uᵢ) of the liver organoids and Uᵢ.

15. The expandable liver organoids of claim 14, wherein the liver tissue-specific gene is LKB1, SLC25A47, SLC13A5, SDS, RDH16, PON3, PON1, MASP2, ITIH4, ITIH3, HSD17B13, HSD11B1, HRG, HPR, HGFAC, HAO1, GNMT, FMO3, F9, CYP4F2, CYP2E1, CYP2D6, CYP1A2, CP, CFHR1, C9, C6, C5orf27, AQP9, APOF, ADH6, APOC4, SERPIND1, SULT2A1, MAT1A, TDO2, SERPINC1, ITIH1, AHSG, C8B, SAA4, LEAP2, HAMP, FMO5, CYP2A6, CFHR2, AGXT, C8G, ALB, APOH, SLC22A1, LECT2, ADH1A, ADH4, SLC38A4, AFM, SLC10A1, CYP8B1, CXCL2, SLCO1B1, UGT2B15, UGT2B10, TAT, SLC2A2, SERPINA6, SERPINA4, HABP2, GC, F12, CYP2C9, CYP2C8, CPB2, CFHR3, BAAT, AKR1C4, APCS, VTN, UGT2B, SERPINA11, PROC, ITIH2, HPX, FGL1, FGG, FGB, FGA, F2, C8A, C4BPB, APOB, APOA2, AMBP, or ANGPTL3.

16. The expandable liver organoids of claim 14, wherein the liver tissue-specific gene is a gene which exhibits the expression amount in the liver tissue at least twice as high as that in other tissues other than the liver tissue.

17. The expandable liver organoids of claim 14, wherein the measurement of the expression amount of the liver tissue-specific gene is the measurement of the mRNA expression level of the gene or the expression level of the protein encoded by the gene.

18. A method for screening hepatotoxic drugs, comprising:
contacting a test substance with the liver organoids of claim 9; and
measuring cell viability or an oxygen consumption rate (OCR) in the liver organoids.

19. A method for screening therapeutic agents for fatty liver, comprising:
producing the liver organoids in claim 9 into fatty liver organoids; and
treating the fatty liver organoids with candidate materials for therapeutic agents for fatty liver.
